(19) United States
(12) Patent Application Publication
(10) Pub. No.: US 2023/0149632 A1
(43) Pub. Date: May 18, 2023

(54) AUTO-INJECTOR AND RELATED METHODS OF USE

(71) Applicant: Regeneron Pharmaceuticals, Inc., Tarrytown, NY (US)

(72) Inventor: Bryan C. Grygus, Clifton Park, NY (US)

(73) Assignee: Regeneron Pharmaceuticals, Inc., Tarrytown, NY (US)

(21) Appl. No.: 18/055,895

(22) Filed: Nov. 16, 2022

Related U.S. Application Data
(60) Provisional application No. 63/264,121, filed on Nov. 16, 2021.

(51) Int. Cl. A61M 5/315; A61M 5/20

(52) U.S. Cl. CPC A61M 5/31571; A61M 5/2053; A61M 2205/3317; A61M 2005/208

(57) ABSTRACT

An injection device may include a housing, a container disposed within the housing, the container enclosing a fluid and having a first end and a second end, a conduit movable relative to the container, wherein the conduit is not in fluid communication with the fluid enclosed by the container while in a first position, and is in fluid communication with the fluid enclosed by the container and configured to deliver the fluid from the container to a patient while in a second position, and a lock that is removable from the housing, the lock having a first portion and a second portion.

### CROSS-REFERENCE TO RELATED APPLICATION

This application claims priority to U.S. Provisional Patent Application No. 63/264,121, filed on Nov. 16, 2021, which is hereby incorporated by reference in its entirety.

### TECHNICAL FIELD

This disclosure is directed to an auto-injector and related methods of use.

### INTRODUCTION

In various available auto-injectors, upon activation by a user, a needle is deployed, and fluid is delivered from the needle into the user. After completion of fluid delivery, the needle may be retracted for user comfort, needle safety, and positive perception of the product. However, many auto-injectors may be inadvertently triggered when dropped or vibrated. Additionally, many auto-injectors may lack suitable control logic for stopping an injection when appropriate.

### SUMMARY OF THE DISCLOSURE

In one aspect, the present disclosure is directed to an injection device. The injection device may include: a housing; a container disposed within the housing, the container enclosing a fluid and having a first end and a second end; a conduit movable relative to the container, wherein the conduit is not in fluid communication with the fluid enclosed by the container while in a first position, and is in fluid communication with the fluid enclosed by the container and configured to deliver the fluid from the container to a patient while in a second position; and a lock that is removable from the housing, the lock having a first portion and a second portion. In a first configuration where the lock is coupled to the housing, the first portion of the lock may be disposed exterior of the housing and the second portion of the lock may be disposed within the housing between the container and the conduit; in the first configuration, the conduit may be prevented from moving into fluid communication with the fluid enclosed by the container by the second portion of the lock; and in a second configuration where the lock is removed from the injection device, the conduit may be able to move into fluid communication with the fluid enclosed by the container.

In another aspect, the injection device may include: a housing; a plunger coupled to the housing and movable relative to the housing; one or more electronics components used during an injection performed by the injection device, the one or more electronics components being formed within an electrical circuit. In a first configuration, a first portion of the plunger may be disposed within the housing, the electrical circuit may be open, and the one or more electronics components may be in a low-power sleep mode; in a second configuration, the plunger may move outward relative to the housing, and the first portion of the plunger may extend exterior of the housing; and in the second configuration, the electrical circuit may be closed, and the one or more electronics components may be transitioned from the low-power sleep mode, to an active mode.

In another aspect, the injection device may include: a housing, wherein the housing includes a curved bottom surface that is concave when viewed from a point external to the housing that is closer to the bottom surface of the housing than a top surface of the housing; a circuit board positioned adjacent to the bottom surface of the housing, wherein the circuit board includes a skin sensor configured to sense a presence of skin in contact with the bottom surface of the housing; and a controller coupled to the circuit board, wherein the controller is configured to initiate an injection by the injection device only after the skin sensor senses the presence of skin in contact with the bottom surface of the housing.

In another aspect, the present disclosure is directed to a method of manufacturing an injection device. The method may include: depositing a first material onto a mold, the first material having a first opacity; depositing a second material around the mold and the first material, the second material having a second opacity that is higher than the first opacity; and positioning a container enclosing a medicament within the injection device and adjacent to a first portion of the injection device formed by the first material.

In another aspect, the injection device may include: a container disposed within the housing, the container having a first end and a second end; a piston configured to move from the first end of the container toward the second end of the container to dispense a medicament from the container; a drive member configured to drive the piston through the container; an emitter configured to emit a beam of light toward the container; a detector positioned on an opposing side of the container from the emitter, wherein the detector is configured to receive the beam of light emitted from the emitter; and a controller coupled to the drive member, the emitter, and the detector. The controller may be configured to: receive a first signal from the detector while the emitter is off, the first signal corresponding to an ambient level of light surrounding the injection device; receive a second signal from the detector while the emitter is on; calculate a difference between light values represented by the first signal and the second signal; and cease operation of the drive member when the difference is less than a threshold value.

In another aspect, the injection device may include: a container disposed within the housing, the container having a first end and a second end; a piston configured to move from the first end of the container toward the second end of the container to dispense a medicament from the container; a drive member configured to drive the piston through the container; an emitter configured to emit a beam of light toward the container; a detector positioned on an opposing side of the container from the emitter, wherein the detector is configured to receive the beam of light emitted from the emitter; and a controller coupled to the drive member, the emitter, and the detector. The controller may be configured to: initiate the drive member and the emitter; receive a first signal from the detector while the emitter is on, the first signal being representative of an amount of light received by the detector; allow for continued operation of the drive member for a first period of time immediately after initiation of the drive member; and cease operation of the drive member upon determining (1) that the amount of light received by the detector is less than a first threshold light value and (2) before the amount of light received by the detector subsequently rises to or above the first threshold light value, that a current of the drive member is greater than a first threshold current value.

In another aspect, the injection device may include: a container disposed within the housing, the having a first end and a second end; a piston configured to move from the first end of the container toward the second end of the container to dispense a medicament from the container; a drive member configured to drive the piston through the container; and a controller coupled to the drive member. The controller may be configured to: maintain a speed of the drive member until a current of the drive member exceeds a first threshold; and after the current of the drive member exceeds the first threshold, reduce a voltage of the drive member to maintain the current of the drive member below a second threshold that is greater than or equal to the first threshold.

### BRIEF DESCRIPTION OF THE FIGURES

The accompanying drawings, which are incorporated in and constitute a part of this specification, illustrate various examples and together with the description, serve to explain the principles of the disclosed examples and embodiments.

Aspects of the disclosure may be implemented in connection with embodiments illustrated in the attached drawings. These drawings show different aspects of the present disclosure and, where appropriate, reference numerals illustrating like structures, components, materials and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is neither limited to any single aspect nor embodiment thereof, nor to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1 is a perspective view of an auto-injector, according to an example of the disclosure.

FIG. 1A is a perspective view of a portion of a housing of an auto-injector according to the disclosure.

FIG. 1B is a perspective view of a portion of a housing of an auto-injector according to the disclosure.

FIG. 2 is a bottom view of an auto-injector according to the disclosure.

FIG. 3 is a side view of an auto-injector, showing an activating switch extending away from a tissue-facing surface, according to the disclosure.

FIG. 3A is a cross-sectional view of an auto-injector, showing an activating switch extending away from a tissue-facing surface, according to the disclosure.

FIG. 3B is a cross-sectional view of an auto-injector, showing an activating switch in a partially depressed position, according to the disclosure.

FIG. 3C is a cross-sectional view of an auto-injector, showing an activating switch in a fully depressed position, according to the disclosure.

FIG. 4 is an exploded view of an auto-injector, according to the disclosure.

FIG. 4A is a schematic illustration of a control system of an auto-injector according to the disclosure.

FIG. 4B is an exploded view of an auto-injector according to the disclosure.

FIG. 4C is a perspective view of a portion of a housing and an electronics board, according to an aspect of the disclosure.

FIG. 5 is an exploded view of a needle mechanism according to the disclosure.

FIG. 5A is a perspective view of a fluid conduit according to the disclosure.

FIG. 5B is a cross-sectional view of a needle of a fluid conduit according to the disclosure.

FIG. 6 is a perspective view of the needle mechanism of FIG. 5 in a first position according to the disclosure.

FIGS. 7-11 are side views of the needle mechanism of FIG. 5.

FIG. 12 is a side cross-sectional view of a portion of an auto-injector according to the disclosure.

FIG. 13 is a side cross-sectional view of a piercing mechanism according to the disclosure.

FIG. 13B is a side cross-sectional view of an auto-injector according to the disclosure.

FIG. 14 is a side cross-sectional view of a piercing mechanism according to the disclosure.

FIG. 15 is a side view of a needle insert switch according to the disclosure.

FIG. 16A is a perspective view of a lock for an auto-injector according to an aspect of the disclosure.

FIG. 16B is a bottom view of an auto-injector and a lock according to the disclosure.

FIG. 16C is a cross-sectional view of an auto-injector and a lock according to the disclosure.

FIG. 16D is a cross-sectional view of an auto-injector and a lock according to the disclosure.

FIG. 16E is a perspective view of a lock for an auto-injector according to an aspect of the disclosure.

FIG. 16F is a side view of a lock for an auto-injector according to an aspect of the disclosure.

FIG. 17 is a bottom view of an electronics board for an auto-injector according to the disclosure.

FIG. 17A is a perspective view of an electronics board for an auto-injector according to the disclosure.

FIGS. 18-20 depict flowcharts of exemplary methods according to the disclosure.

FIGS. 20A and 20B depict graphs relating to electric controls for an auto-injector according to the disclosure.

FIGS. 21-23 depict flowcharts of exemplary methods according to the disclosure.

Again, there are many embodiments described and illustrated herein. The present disclosure is neither limited to any single aspect nor embodiment thereof, nor to any combinations and/or permutations of such aspects and/or embodiments. Each of the aspects of the present disclosure, and/or embodiments thereof, may be employed alone or in combination with one or more of the other aspects of the present disclosure and/or embodiments thereof. For the sake of brevity, many of those combinations and permutations are not discussed separately herein.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art appreciates that the cross-sectional views are not drawn to scale and should not be viewed as representing proportional relationships between different components. The cross-sectional views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. In the discussion that follows, relative terms such as “about,” “substantially,” “approximately,” etc. are used to indicate a possible variation of ±10% in a stated numeric value.

As described above, existing auto-injectors may be inadvertently triggered when dropped or vibrated. Additionally, existing auto-injectors may lack suitable control logic for stopping an injection when appropriate. These shortcomings may cause premature deployment of drugs, increase complexity of self-administration of drugs, introduce user errors, and cause user discomfort. Accordingly, the present disclosure is directed to various embodiments of an injection device (e.g., auto-injector) for self-administration of drugs, or other therapeutic agents, by a user. Specifically, according to certain embodiments, a likelihood of inadvertent triggering of the auto-injector may be reduced and the auto-injector may further incorporate control logic which improves operation of the auto-injector and user experience.

Additional details of auto-injectors in accordance with the present disclosure can be found in PCT/US2018/031077 to Arnott, et al., filed on Nov. 8, 2018, and published as WO 2018/204779 A1, the entirety of which is incorporated by reference herein. Additional details of vial piercing systems in accordance with the present disclosure can be found in U.S. Pat. No. 10,182,969, filed on Mar. 10, 2016, the entirety of which is incorporated by reference herein.

Overall System

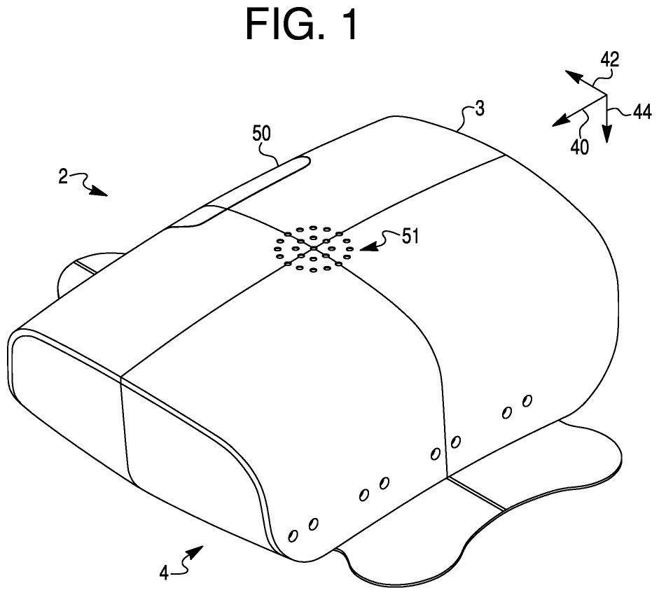

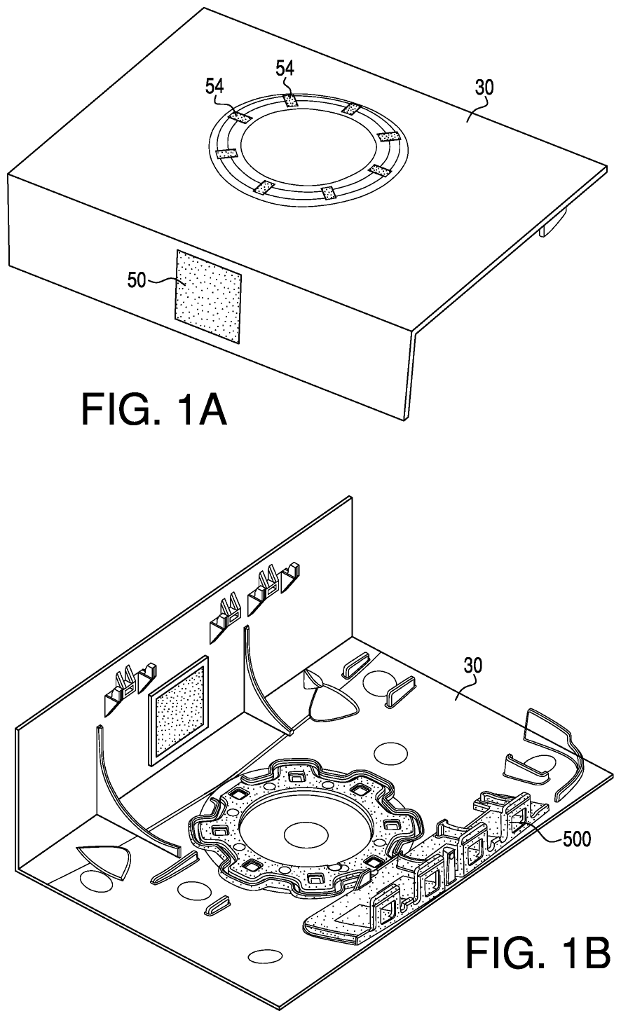

An example of such an auto-injector 2 is shown in FIGS. 1, 2, and 3. As shown in FIG. 1, auto-injector 2 may include a housing 3 having a tissue-engaging (e.g., bottom) surface 4 through which a needle may be deployed and retracted. As shown in FIGS. 1 and 1A, housing 3 may include a transparent window 50. Transparent window 50 may enable a viewer to visualize one or more displays or to visualize an interior of auto-injector 2 and components therein, such as a primary container and/or a drug product stored in the primary container.

In some embodiments, and as shown in FIG. 1, auto-injector 2 may include a plurality of openings 51 configured to facilitate the travel of sound generated within housing 3 (by, e.g., a speaker). Auto-injector 2 may have any suitable dimensions to enable portability and self-attachment by a user. In one example, auto-injector 2 may have a length of about 2.98 inches, a width of about 2.07 inches, and a height of about 1.07 inches. However, other suitable values also may be utilized, including, e.g., a length from about 0.5 inches to about 5.0 inches, a width of about 0.5 inches to about 3.0 inches, and a height from 0.5 inches to about 2.0 inches.

Auto-injector 2 may be oriented about a longitudinal axis 40 (e.g., an X axis), a lateral axis 42 (e.g., a Y axis) that is substantially perpendicular to longitudinal axis 40, and a vertical axis 44 (e.g., a Z axis) that is substantially perpendicular to both longitudinal axis 40 and lateral axis 42.

As shown in FIG. 1, an adhesive patch 12 may be coupled to tissue-engaging surface 4 to help secure auto-injector 2 to a user's body (e.g., skin). Adhesive patch 12 may be formed from fabric or any other suitable material, and may include an adhesive. The adhesive may be an aqueous or solvent-based adhesive, or may be a hot melt adhesive, for example. Suitable adhesives also include acrylic based, dextrin based, and urethane based adhesives as well as natural and synthetic elastomers. In some examples, the adhesive provided on patch 12 may be activated upon contact with a user's skin. In yet another example, patch 12 may include a non-woven polyester substrate and an acrylic or silicone adhesive. Patch 12 may be joined to housing 3 by, e.g., a double-sided adhesive, or by other mechanisms like ultrasonic welding. Patch 12 may have a length dimension greater than a width of auto-injector 2.

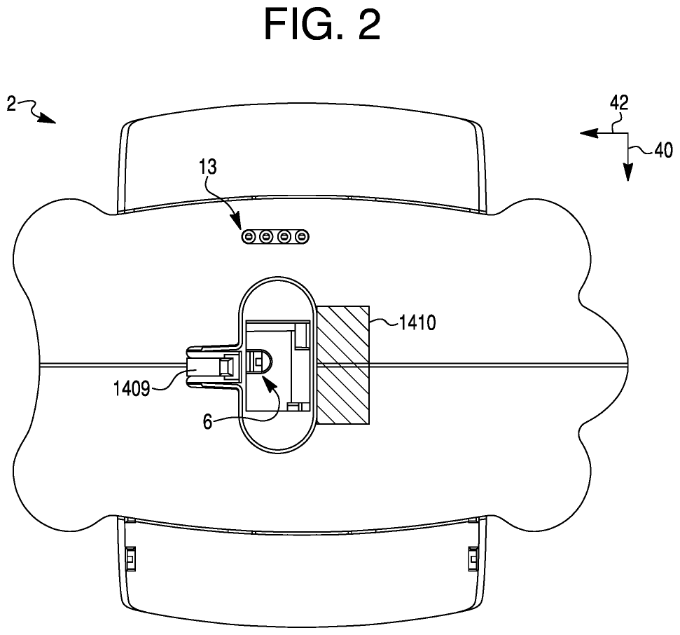

As shown in FIG. 2, auto-injector 2 may include an opening 6, through which the needle may be deployed and retracted. An activating switch 1409 may be disposed on tissue-engaging surface 4, and may be configured to activate auto-injector 2, or otherwise place auto-injector 2 in a “ready” mode. A touch sensor 1410 also may be disposed on tissue-engaging surface 4, and may be configured to help a controller of auto-injector 2 determine whether auto-injector 2 is disposed on the skin of a user (indicating that the auto-injector should fire or otherwise deploy a needle), or whether activating switch 1409 was improperly triggered (indicating that operation of auto-injector 2 should be stopped). A connecting port 13 also may be disposed on tissue-engaging surface 4 to facilitate programming of auto-injector 2.

Auto-injector 2 may be configured to operate in three or more operation phases including, e.g., an injection sequence activation phase, an injection phase, and a retraction phase, each of which will be described in further detail herein. The injection sequence activation phase, injection phase, and retraction phase may collectively be referred to herein as an “injection sequence.”

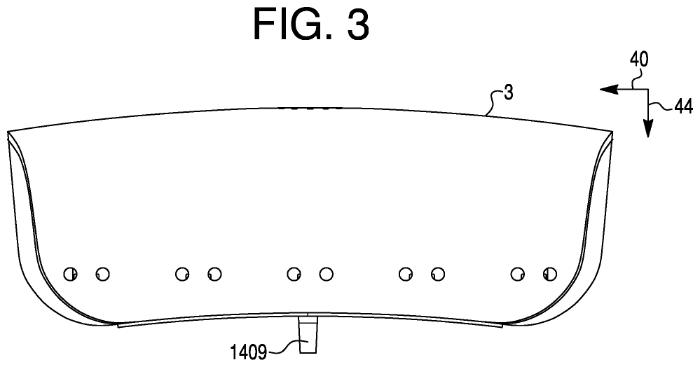

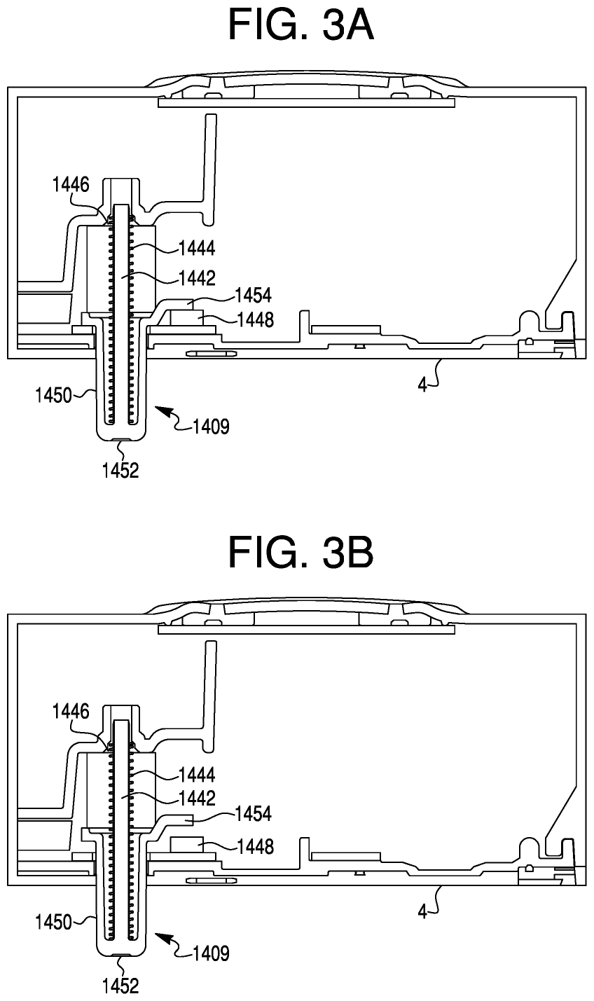

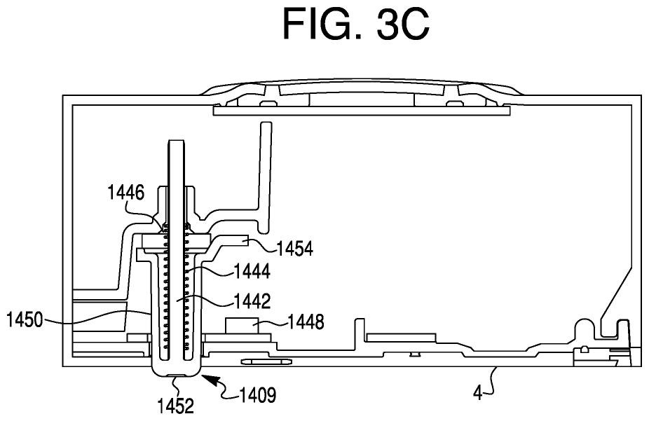

Referring to FIGS. 3A, 3B, and 3C which show cross-sections of an auto-injector 2, activating switch 1409 may be a mechanical plunger-type switch. For example, activating switch 1409 may include a plunger 1450 having a plunger contact surface 1452. Plunger contact surface 1452 may be generally circular in shape (or have another suitable shape) and may be large enough to be depressed comfortably by soft skin. In some embodiments, plunger contact surface 1452 may have a diameter or width ranging from about 2 mm to about 10 mm, a diameter ranging from about 4 mm to about 8 mm, or a diameter of about 6 mm. Activating switch 1409 may further include a shaft 1442, a biasing member 1444, a biasing collar 1446, and a plunger flange 1454. Biasing member 1444 may be a spring, for example, and may surround shaft 1442. Biasing member 1444 may be fixed, or otherwise prevented from moving, at one end by biasing collar 1446. Plunger flange 1454 may be configured to contact or otherwise depress a plunger switch 1448. For clarity, the term activating switch and/or reference to activating switch 1409, as used herein, should be understood to encompass any or all components of activating switch 1409, including shaft 1442, biasing member 1444, biasing collar 1446, plunger switch 1448, plunger 1450, plunger contact surface 1452, and plunger flange 1454.

In a free state, i.e., when plunger 1450 is not depressed, either by being pressed against the skin of a user or otherwise, plunger 1450 may extend outwardly from tissue-engaging surface 4 as shown in FIG. 3A. In the free state, plunger contact surface 1452 may be a distance from tissue-engaging surface 4 ranging from about 1 mm to about 16 mm, ranging from about 5 mm to about 12 mm, or a distance of about 8.5 mm. In the free state, biasing member 1444 may urge plunger 1450 to extend outwardly from tissue-engaging surface by pressing against biasing collar 1446. In the free state, plunger flange 1454 may be in contact with, or otherwise depress plunger switch 1448. When plunger flange 1454 is in contact with, or otherwise depresses plunger switch 1448, an electrical circuit associated with plunger switch 1448 may be complete, or closed.

When plunger 1450 is depressed either by being pressed against the skin of a user or otherwise, plunger 1450 may initially move to a partially depressed state, as shown in FIG. 3B. In the partially depressed state, biasing member 1444 may be compressed against biasing collar 1446. Plunger flange 1454 may further be out of contact with, or otherwise not depressing plunger switch 1448. When plunger flange 1454 is spaced apart from, not in contact with, or is otherwise not depressing plunger switch 1448, the electrical circuit associated with plunger switch 1448 may be broken, or open. By this configuration, the auto-injector 2 may be maintained in a reduced power state while plunger 1450 is depressed, such as when auto-injector 2 is in packaging.

As shown in FIG. 3B, plunger 1450 may not necessarily travel to the fully depressed state (shown in FIG. 3C) before plunger flange 1454 is out of contact with plunger switch 1448. As shown in FIG. 3C, on the other hand, in the fully depressed state, plunger 1450 may be depressed inwardly such that plunger contact surface 1452 is flush or nearly flush with tissue-engaging surface 4.

Plunger flange 1454 may be out of contact with plunger switch 1448, for example, after less than 5 mm of travel by plunger 1450, after less than 3 mm of travel by plunger 1450, after less than 1 mm of travel by plunger 1450, or after about 0.75 mm of travel by plunger 1450—all when, for example, the maximum depression distance is 8.5 mm. In other words, plunger 1450 may transition from the free state, in which plunger flange 1454 is in contact with plunger switch 1448, to the partially depressed state, in which plunger flange 1454 is out of contact with plunger switch 1448, after moving only a portion of a maximum depression distance of plunger 1450 relative to housing 3 of auto-injector 2. For example, plunger 1450 may transition to the depressed state after moving only about 5%, about 10%, or about 20% of the maximum depression distance. Accordingly, the auto-injector 2 and plunger switch 1448 may be sufficiently responsive upon depressing plunger 1450 against a user's skin. For example, auto-injector 2 and plunger switch 1448 may be sufficiently responsive when pressed against skin of varying firmness or users having varying body fat content. While examples of travel distances for plunger 1450 are provided herein, it is to be understood that the present disclosure is not limited to any particular examples and any suitable travel distance may be used.

Biasing member 1444 may be sufficiently stiff such that in the free state, plunger flange 1454 stays in contact with or otherwise continuously depresses plunger switch 1448. Biasing member 1444 may also be of a stiffness such that plunger 1450 may be depressed comfortably when pressed against a user's skin. Biasing member 1444 may be biased to maintain plunger 1450 in the free state.

Though activating switch 1409 is shown in FIGS. 3A-3C as a mechanical plunger-type switch, it is to be understood that activating switch 1409 may be any other suitable type of switch, such as a rocker switch, throw switch, toggle switch, temperature switch, and the like. Additionally, while the electrical circuit associated with plunger switch 1448 is described herein as closed when plunger 1450 is in the free state and open when plunger 1450 is in the depressed state, it should be understood that an opposite configuration may be used. For example, the electrical circuit associated with plunger switch 1448 may be open when plunger 1450 is in the free state and closed when plunger 1450 is in the depressed state.

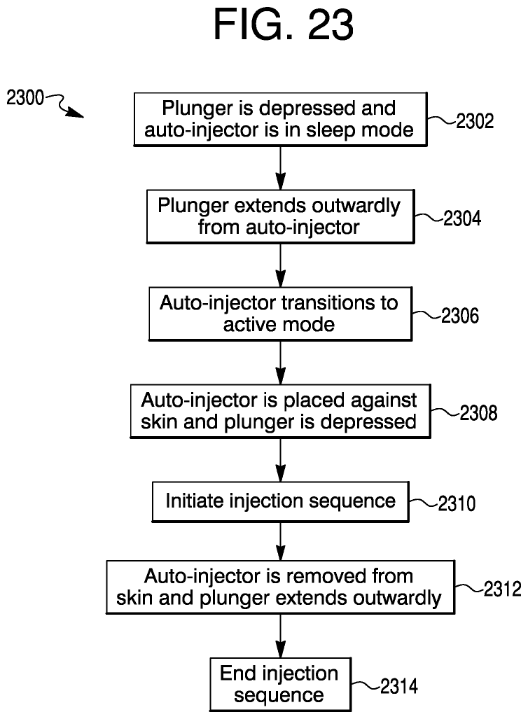

A method of controlling auto-injector 2 according to positions of activating switch 1409 will be described hereinafter in further detail with reference to FIG. 23.

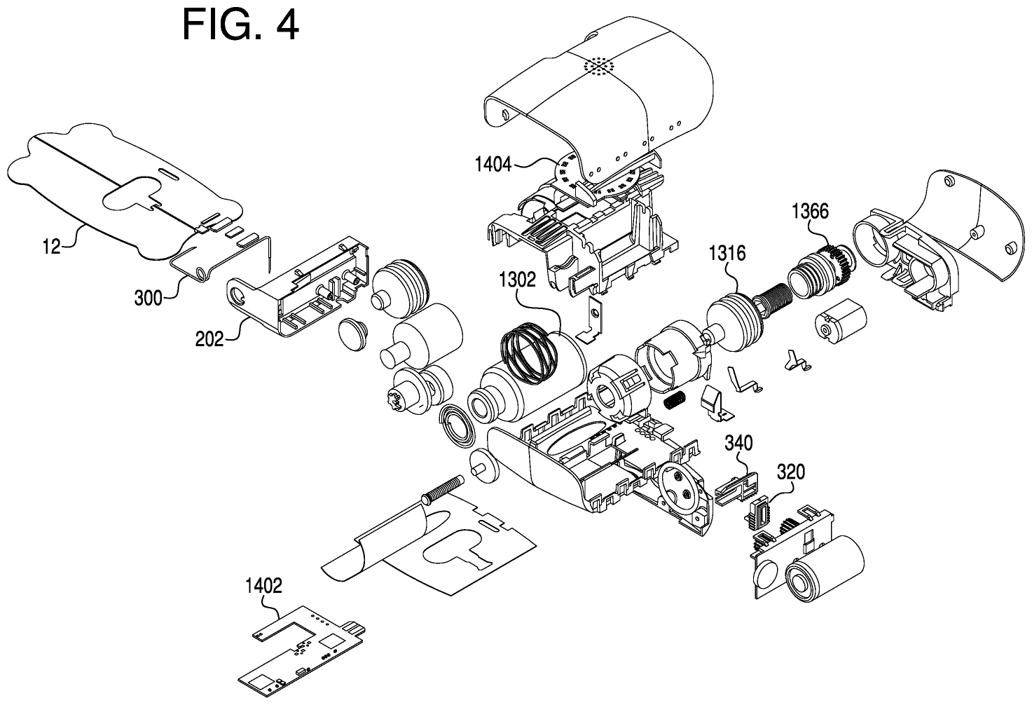

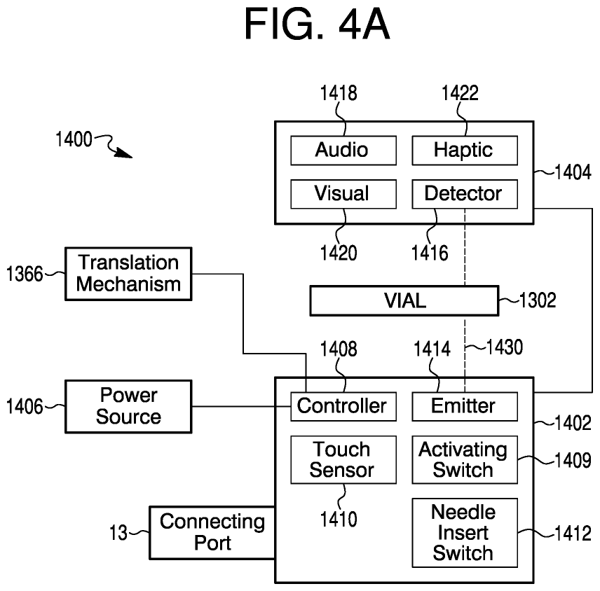

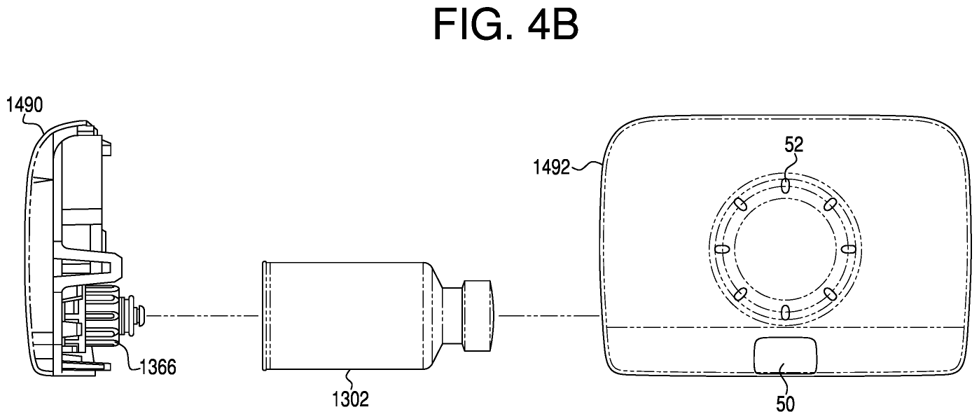

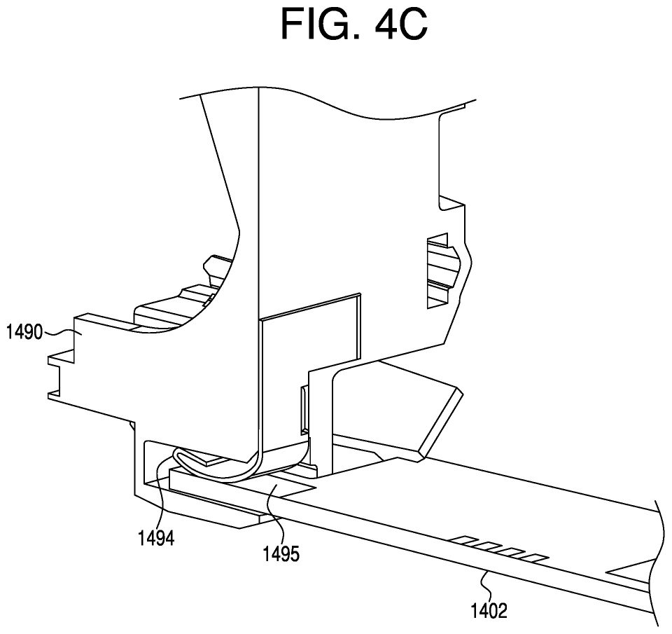

Further, as shown in FIG. 4B, in some embodiments, auto-injector 2 may include a plurality of LEDs 52. The LEDs 52 may be arranged in a ring-like formation, or any other suitable formation. As described in further detail hereinafter, light from the one or more LEDs 52 may be indicative of various operational states of the auto-injector 2.

Auto-Injector Housing

Referring to FIGS. 1A and 1B, the housing 3 of auto-injector 2 may include an upper portion 30. The upper portion 30 may form a portion of the housing 3 opposite tissue-engaging surface 4. The upper portion 30 may include transparent window 50 through which a user may be able to see contents of the auto-injector 2, including a vial and/or a drug contained in the vial. The transparent window 50 may be positioned on a side of the upper portion 30 and may be formed such that it conforms to a rounded/curved contour of the upper portion 30, as shown in FIG. 1. The transparent window 50 may be generally rectangular in shape with rounded corners.

The upper portion 30 may also include a plurality of transparent windows 54. The transparent windows 54 may be formed on a top surface of upper portion 30 and may be arranged in any suitable configuration, such as a circular configuration, an oval configuration, a rectangular configuration, or a linear configuration, for example. Transparent windows 54 may be circumferentially spaced apart from one another, for example. The transparent windows 54 may allow light from one or more LEDs located within the housing 3 to be visible to a user. The light from the one or more LEDs may be indicative of various operational states of the auto-injector 2, as described herein.

The transparent window 50 and the transparent windows 54 may be integrally formed as part of the upper portion 30. As shown in FIG. 1B, the upper portion 30 may include a transparent portion 500 formed from a transparent material. The transparent portion 500 may be contiguous, such that the transparent window 50 and the transparent windows 54 are formed of one piece of transparent material. Moreover, the transparent portion 500 may be integrated into the upper portion 30 such that the upper portion 30, including the transparent window 50 and the transparent windows 54 is manufactured as a single part.

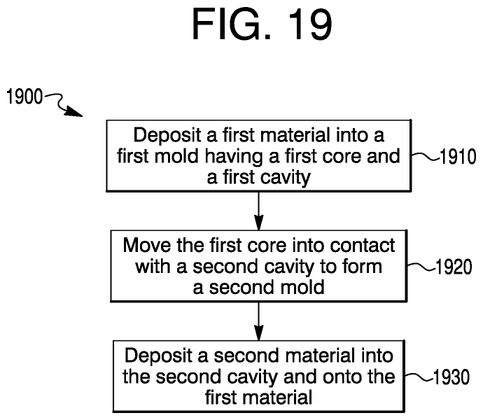

To form the upper portion 30 as a single part, the upper portion 30 may be manufactured using a double shot molding process, for example. FIG. 19 illustrates an exemplary method 1900 of molding the upper portion 30 using double shot molding or insert molding. At step 1910, a first material may be deposited into a first mold having a first core and a first mold cavity. The first material may have a low opacity and may be, for example, a transparent material for the transparent window 50 and the transparent windows 54. The first material may be, for example, transparent acrylic, clarified acrylonitrile butadiene styrene (ABS), polycarbonate, polyvinylchloride (PVC), or polyethylene terephthalate glycol (PETG). The first mold may be configured, for example, to form transparent portion 500.

At step 1920, the first core and the material in the first mold cavity, may be moved within a second mold cavity to form a second mold. When moved, the first core may retain the first material. The second mold may be configured, for example, to form the upper portion 30. At step 1930, a second material may be deposited into the second mold cavity in which the first material is contained. The second material may be deposited around the first material and first core in unoccupied space of the second mold cavity to form the upper portion 30. The second material may be a material with high opacity, such as white plastic. The second material may be, for example, ABS, polycarbonate, ABS-polycarbonate blend, PVC, or PETG.

Accordingly, the generally opaque upper portion 30, which includes the transparent window 50 and the transparent windows 54 may be formed from two different materials to form a single part. Forming the upper portion 30 as a single part may reduce an overall number of steps required to assemble auto-injector 2. For example, in some embodiments, no fastening or adhesive steps or materials are needed to join transparent and opaque portions of the housing. Avoiding unnecessary assembly steps may further improve the appearance of cosmetic surfaces of the auto-injector 2. Additionally, forming the upper portion 30 as a single part may improve the overall structural integrity of the auto-injector 2. Further, forming the upper portion 30 as a single part may reduce or eliminate sinks on cosmetic surfaces.

Needle Mechanism

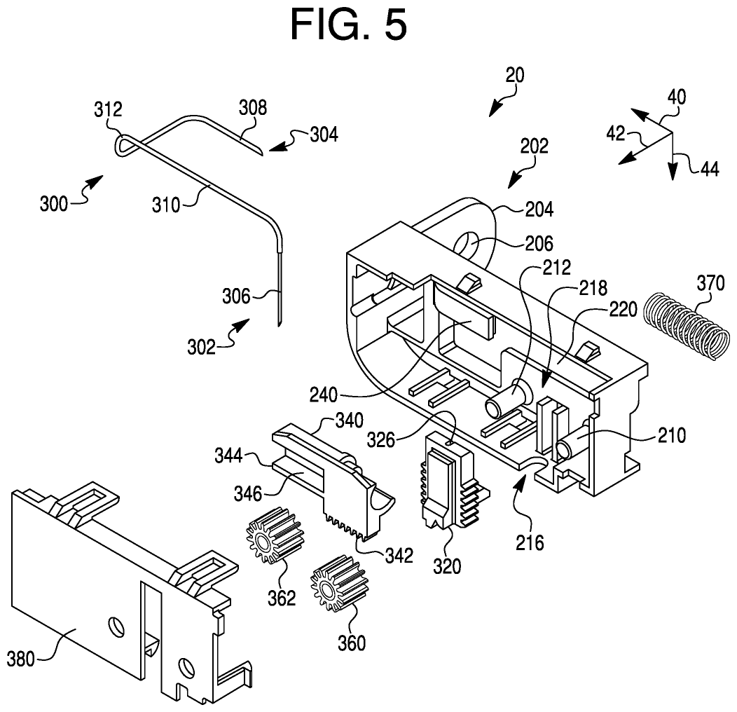

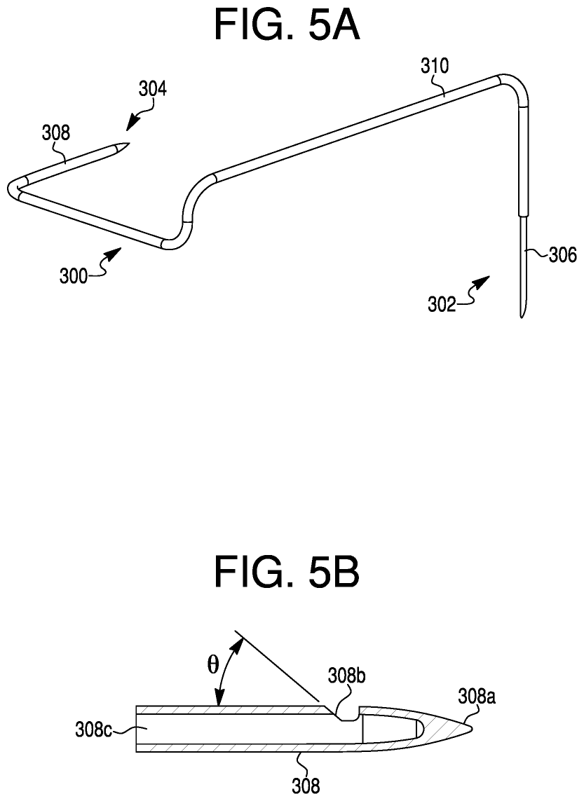

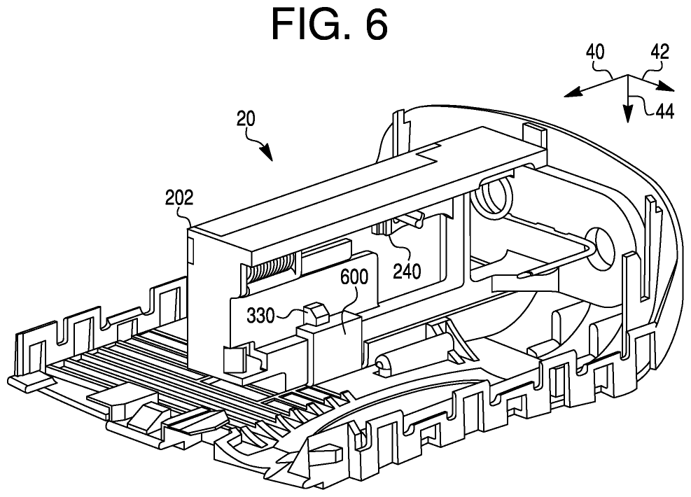

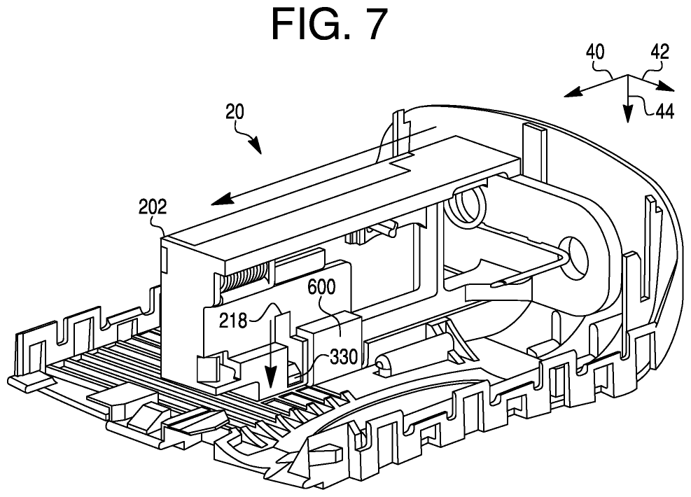

Referring to FIGS. 5-11, a needle mechanism 20 includes a carrier 202 that is movable (e.g., slidable) within housing 3 between a first position (FIG. 6) and a second position (FIG. 7). Needle mechanism 20 also may include a fluid conduit 300 that is mounted to carrier 202, and which may be deployed into a user, and retracted by a driver 320. A shuttle 340 (e.g., a shuttle actuator) may be configured to move driver 320 via a deployment gear 360, and a retraction gear 362. Shuttle 340 may be coupled to a resilient member (e.g., a spring 370). A cover 380 (FIG. 5) may be coupled to carrier 202 to enclose various components of needle mechanism 20.

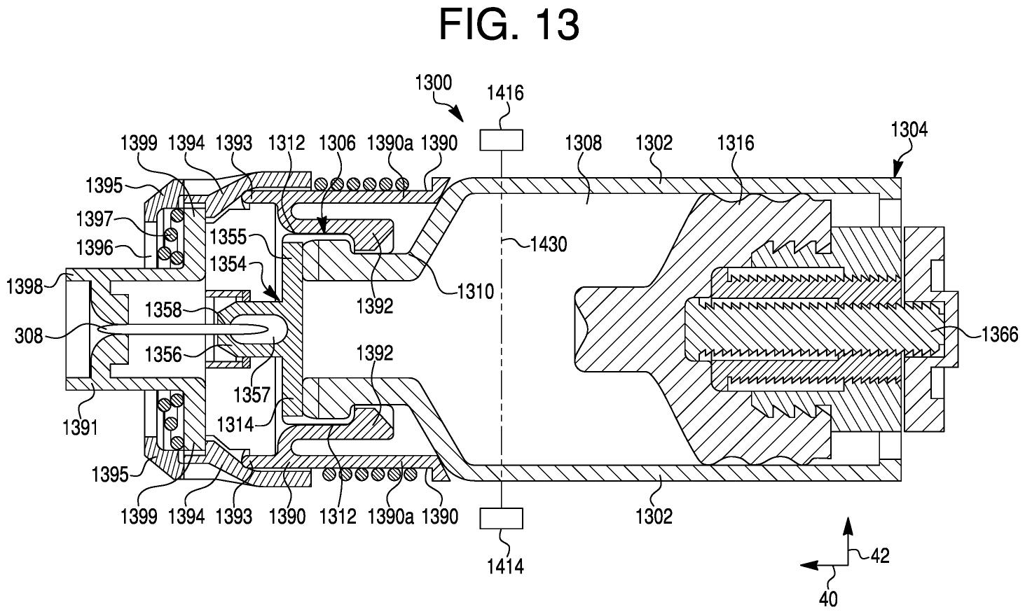

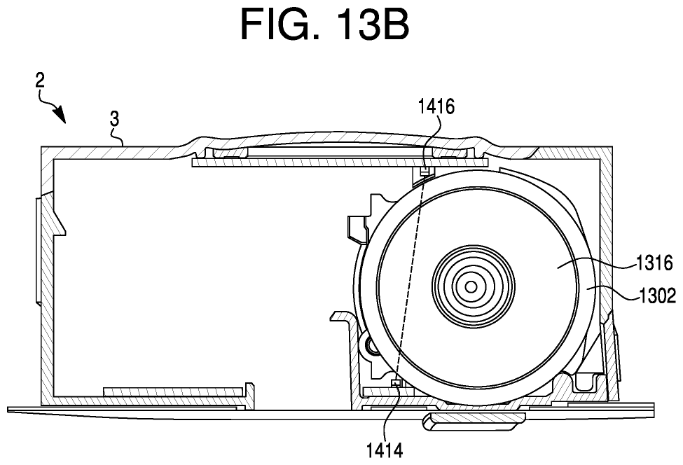

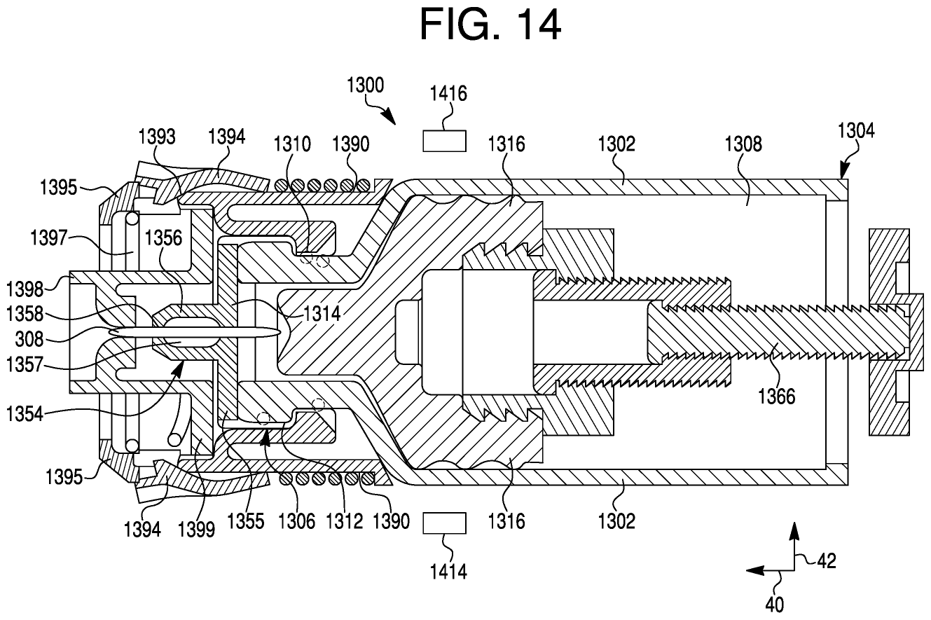

Referring to FIG. 5, fluid conduit 300 may extend from a first end 302 to a second end 304. As shown in further detail in FIG. 5A, first end 302 may include a needle 306 that is configured to be injected into a user. Needle 306 may include a sharp and/or beveled tip, and may extend generally along or parallel to axis 44. Second end 304 may include a needle 308 that is substantially similar to needle 306, but may be positioned within auto-injector 2 to penetrate a cartridge 1302 (shown in FIG. 13 and described in further detail below) to access drugs to be injected into the user. Fluid conduit 300 may include an intermediate section 310 including one portion extending along or parallel to axis 40, and a second portion extending along or parallel to axis 40. The first and second portions of intermediate section 310 may be joined in a serpentine section 312 that facilitates flexion of fluid conduit 300 and movement of needle 306 along axis 44 during deployment into the user, and during retraction out of the user. While a serpentine section 312 is shown, any other suitable shape, e.g., a coil, curved, or other shape that enables flexion of fluid conduit 300 is also contemplated. Serpentine section 312, or similar structure, may act as a cantilever when needle 306 is deployed and/or retracted. Serpentine section 312 also may bias fluid conduit 300 into the deployed configuration shown in FIG. 5. Once needle 308 penetrates and establishes fluid communication with cartridge 1302 (see, e.g., FIG. 14), drugs may travel from cartridge 1302, through needle 308, intermediate section 310, and needle 306 (pierced through the user's skin), and into the user. In some examples, fluid conduit 300 may include only metal or a metal alloy. In other examples, fluid conduit 300 may be any other suitable material, such as, e.g., polymers or the like. Needle 308 and intermediate portion 310 may define a 22 or 23 Gauge, thin-walled needle, while needle 306 may be a 27 Gauge, thin-walled needle. Other needle sizes ranging from, e.g., 6 Gauge to 34 Gauge, and other needle wall thicknesses, such as regular wall, extra-thin wall, and ultra-thin wall also may be utilized as appropriate. Fluid conduit 300 may reduce the amount of material that contacts the drugs, reduce joints and assembly steps, and require less sterilization than conventional devices.

As shown in FIG. 5B, needle 308 may be configured to include a needle tip 308a and a side port 308b. Side port 308b may be fluidly connected to fluid path 308c and allow fluid to enter fluid conduit 300 through a side of needle 308, as opposed to through the tip of needle 308. A rear wall of side port 308b may be inclined at an angle θ relative to a longitudinal axis of fluid path 308c. In some embodiments, the angle θ may be between about 20° and 60°, between about 30° and 50°, or about 40°. By configuring needle 308 in this way, needle 308 may be optimized for piercing the primary container of auto-injector 2, which may be a sealed cartridge or a vial. The relative positioning of needle tip 308a and side port 308b may allow piercing of the seal of the primary container without coring or otherwise cutting a portion of the seal with an opening to fluid path 308c. Thereby, entry of particles cored or cut from the seal into the fluid path 308c may be minimized or avoided.

Needle 306 may be configured substantially similarly to needle 308, as shown in FIG. 5B. Alternatively, in some embodiments, either or both of needles 306 and 308 may be a 3 bevel needle, a 5 bevel needle, or any other suitable type of needle. In some embodiments, one or both of needles 306 and 308 may be a pencil point needle having a round hole or any other suitably shaped hole.

Carrier 202 may be formed of plastic (e.g., injection-molded plastic), a metal, metal alloy, or the like, and may include a flange 204 with an opening 206, and posts 210 and 212. Carrier 202 also may include an opening 216 through which a needle or other fluid conduit may be deployed. Opening 216 may be a slot that is recessed from an end surface of carrier 202, or, in an alternative embodiment, an entirety of the perimeter of opening 216 may be defined by material of carrier 202. Carrier 202 also includes a driver path 218. Driver path 218 may be a slot in carrier 202 that extends along or parallel to axis 44. Driver path 218 may be configured to receive a protrusion of driver 320, such as, e.g., protrusion 330 discussed in further detail below. Carrier 202 also may include a shuttle path 220, along which shuttle 340 may move, as described in further detail below.

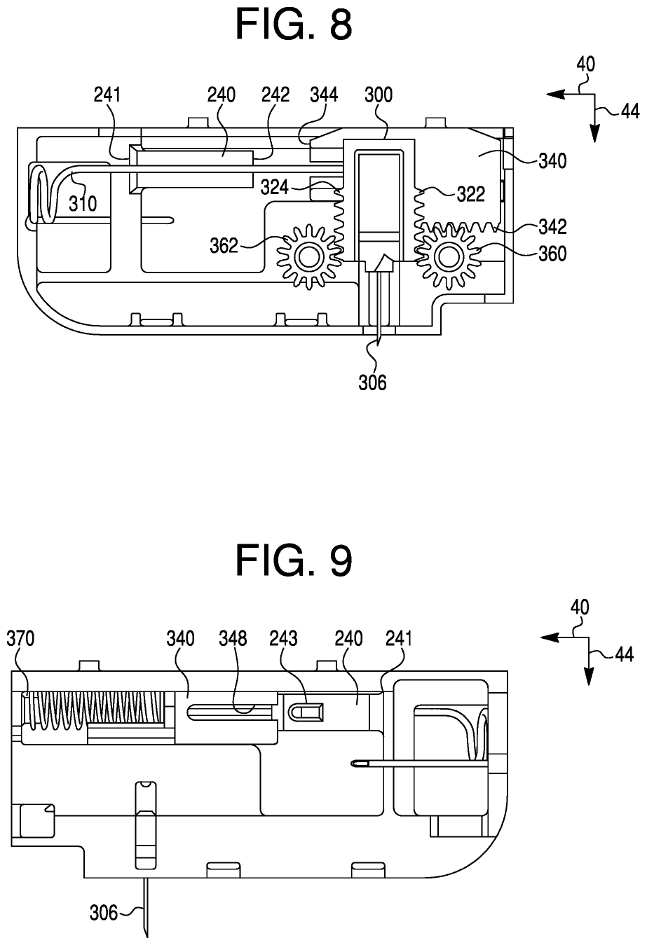

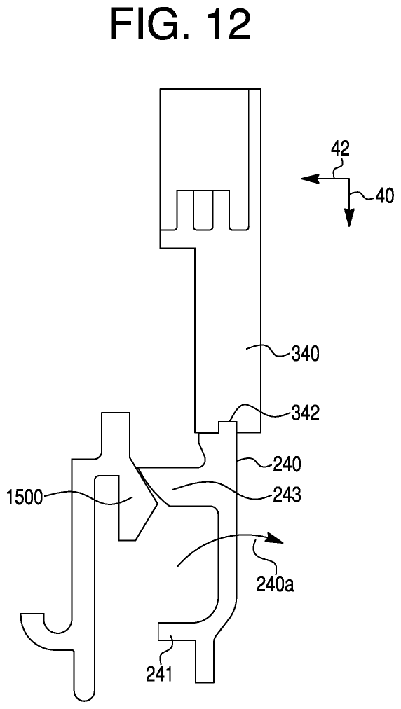

Carrier 202 also may include a stop 240 that is configured to engage shuttle 340. Stop 240 may be a cantilever having a fixed end 241 (FIG. 8) and a free end 242 (FIG. 8). Stop 240 may include an inclined ramp 243 (FIGS. 9 and 12) that, when engaged or pushed by a ramp 1500 (described with reference to FIG. 12), causes stop 240 to deflect about fixed end 241. In a first position, free end 242 may block or otherwise impede movement of shuttle 340, and in a second configuration, may permit movement of shuttle 340. The relationship between stop 240 and shuttle 340 will be discussed in further detail later in the application.

Driver 320 includes two racks 322 and 324 (shown in FIG. 8) parallel to one another and disposed on opposing sides of driver 320. Racks 322 and 324 may include teeth and may be configured to engage with and drive rotation of deployment gear 360 and retraction gear 362, respectively. Driver 320 may include a lumen 326 (or a track, recess, or other suitable structure) (FIG. 5) that is configured to receive needle 306 of fluid conduit 300. Driver 320 also may include protrusion 330 (FIGS. 6 and 7) that is configured to slide within driver path 218 of carrier 202. Protrusion 330 may include a hook-like configuration that can “catch” on impediment 600, as described in further detail below.

With continuing reference to FIG. 5, shuttle 340 may include a rack 342 configured to engage with gears 360 and 362. Shuttle 340 also may include an end surface 344, and a recess 346 that extends along a length of shuttle 340 in the same direction as rack 342. A slot 348 (FIG. 9) may extend along the length of recess 346. Slot 348 may extend through the middle of recess 346 and may extend along an entirety or substantial entirety of recess 346.

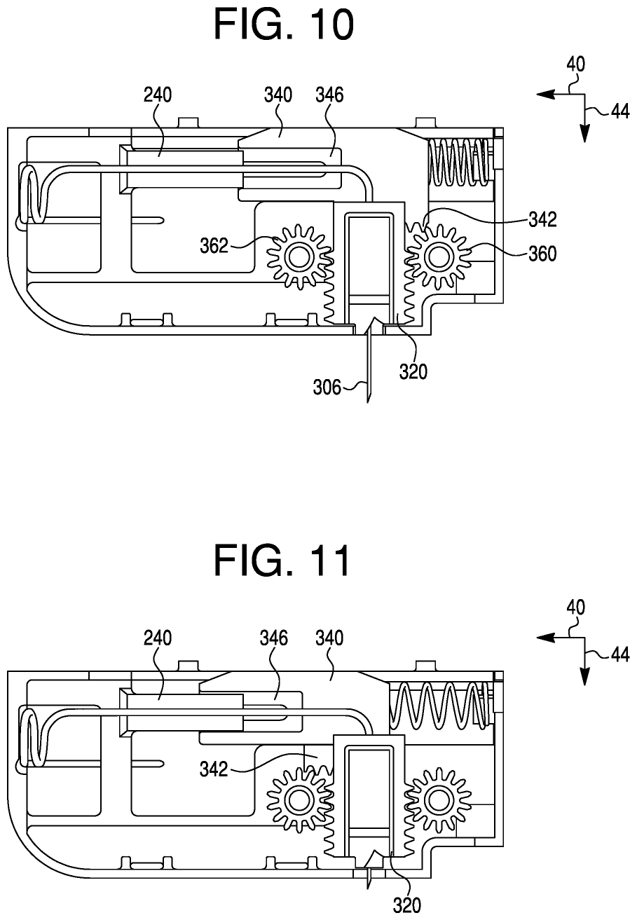

Shuttle 340 may move along track 220 from a first, starting position (FIG. 8), to a second, intermediate position (FIGS. 9 and 10), and from the second position to a third, final position (shown between the second and third configurations in FIG. 11). As shuttle 340 moves along track 220, rack 342 may first engage deployment gear 360, and then retraction gear 362. At certain times, rack 342 engages at most one of deployment gear 360 and retraction gear 362 at any given time. In some examples, such as when rack 342 is disposed longitudinally between deployment gear 360 and retraction gear 362, rack 342 is not engaged with either of deployment gear 360 and retraction gear 362. Shuttle 340 may be configured to move only along one axis (e.g., axis 40) and only in one direction along the one axis. The force required to move shuttle 340 along track 220 may be provided by expansion of spring 370. Spring 370 may be compressed from a resting state, and the expansion of spring 370 may move shuttle 340 along track 220 through the series of positions/configurations set forth above. At various positions of shuttle 340, different features of auto-injector 2 may directly or indirectly block movement of shuttle 340. Alternatively, it is contemplated that spring 370 may be biased to a compressed configuration. In this alternative embodiment, spring 370 may be expanded from a resting state, and compression of spring 370 may move shuttle 340 along track 220 through the series of positions/configurations set forth above.

The first position of shuttle 340, shown in FIG. 8, may correspond to an unused, undeployed, and/or new state of auto-injector 2. In this first position, driver 320 may be in an undeployed state. Shuttle 340 is maintained in the first position by the positioning of an impediment 600 in the path of driver 320 (FIG. 6). Impediment 600, which may be a shelf of housing 3, or another suitable blocking device, may prevent movement of driver 320 by engaging and/or retaining protrusion 330. Therefore, because driver 320, deployment gear 360, and rack 342 are coupled to one another, the blockage of driver 320 also prevents movement of shuttle 340. Shuttle 340 may move from the first position to the second position by moving impediment 600 relative to carrier 202 (or vice versa). In one example, carrier 202 is moved (e.g., to the left in FIG. 6) while impediment 600 remains stationary.

When the path of driver 320 is free from impediment 600 (FIG. 7), spring 370 may expand and move shuttle 340 along track 220. This linear movement of shuttle 340 may rotate deployment gear 360 counter-clockwise (or clockwise in other examples) via rack 342, and the rotation of deployment gear 360 may move driver 320 downward along axis 44, via rack 322 of driver 320. This downward movement of driver 320 may cause needle 306 to pierce through the skin of a user. In some examples, driver 320 may be configured to move, relative to carrier 202, along only axis 44.

Shuttle 340 may be moved by the expansion of spring 370 until its end surface 344 abuts free end 242 of stop 240 such that shuttle 340 is maintained in the second position shown in FIGS. 9 and 10. At this point, free end 242 may prevent further expansion of spring 370 and further movement of shuttle 340 along track 220. In this second position, fluid conduit 300 may be deployed within a user, and fluid from cartridge 1302 may be injected into the user via needle 306. Additionally, while shuttle 340 is in the second position, rack 342 may be engaged with deployment gear 360 to maintain needle 306 in the deployed configuration. Shuttle 340 may move from the second position to the third position by the flexion of stop 240 about its fixed end 241. Further details of this flexion are set forth below with respect to FIGS. 12-14. The flexion of stop 240 may allow spring 370 to continue expanding, urging shuttle 340 further along track 220. In some examples, stop 240 may be received by and/or within recess 346 of shuttle 340, and ramp 243 may slide within slot 348, as shuttle 340 moves from the second position to the third position.

The movement of shuttle 340 from the second position to the third position may correspond to the retraction of needle 306 from the user into housing 3. In particular, rack 342 may engage with and rotate retraction gear 362 in the same direction (e.g., counter-clockwise or clockwise) as deployment gear 360 was rotated. The rotation of retraction gear 362 may urge driver 320 back to a retracted position via rack 324. Shuttle 340 may reach the third position, where driver 320 is fully-retracted, when its end surface 344 engages a wall of carrier 202, when free end 242 of stop 240 reaches an end of recess 346, and/or when spring 370 reaches a resting state.

In some embodiments, once driver 320 moves from the deployed state back to the retracted state, it may be prevented from moving out of the retracted state. As a result, needle 306 will be prevented from re-deployment into the user. In this configuration, auto-injector 2 may be a single-use device (e.g., discarded after completing one injection). In other embodiments, auto-injector 2 may be reset and reused. Furthermore, deployment gear 360 and retraction gear 362 may be the only rotating gears disposed within auto-injector 2, in some examples.

Piercing System and Sterile Connector

FIGS. 13 and 14 show features of a piercing system 1300 of auto-injector 2. Additional details of exemplary piercing systems can be found in U.S. Patent Application Publication No. 2016/0262984 A1 to Arnott et al., published on Sep. 15, 2016, the entirety of which is incorporated by reference herein. Piercing system 1300 includes a primary container, which may be a cartridge 1302 with a first end 1304 and a second end 1306. The primary container may alternatively be a chamber, syringe, vial, flexible sac, or any other suitable fluid containing structure.

Cartridge 1302 may include a cavity 1308 opened at first end 1304 and extending toward second end 1306. Second end 1306 may include a neck 1310 with a cap 1312 that engages neck 1310 to close second end 1306. A septum 1314 may be positioned between cartridge 1302 and cap 1312 to assist with closing second end 1306, and allow for needle 308 (e.g., a staked needle) to be inserted into cartridge 1302. Cavity 1308 may be closed at first end 1304 by a piston 1316.

Cartridge 1302 may have a 5 mL capacity in some examples, although any other suitable volume (e.g., from 1 mL to 50 mL, or from 2 mL to 10 mL, or from 3 mL to 6 mL, or from 2 mL to 5 mL, or another suitable range) also may be utilized depending on the drug to be delivered. In other examples, cartridge 1302 may have a capacity greater than or equal to 1 mL, or greater than or equal to 2 mL, or greater than or equal to 3 mL, or greater than or equal to 4 mL, or greater than or equal to 5 mL, or greater than or equal to 10 mL, or greater than or equal to 15 mL. Cartridge 1302 may contain and preserve a drug for injection into a user, and may help maintain sterility of the drug. Cartridge 1302 may have a 13 mm diameter neck, a 45 mm length, and an internal diameter of 19.05 mm. These values are merely exemplary, and other suitable dimensions may be utilized as appropriate. In some examples, cartridge 1302 may be formed using conventional materials, and may be shorter than existing devices, which can help auto-injector 2 remain cost-effective and small. Cartridge 1302 may be a shortened ISO 10 mL cartridge.

Septum 1314 may include an uncoated bromobutyl material, or another suitable material. Piston 1316 may include a fluoropolymer coated bromobutyl material, and also may include a conical nose 1316a to help reduce dead volume within cartridge 1302. Piston 1316 may include one or more rubber materials such as, e.g., halobutyls (e.g., bromobutyl, chlorobutyl, florobutyl) and/or nitriles, among other materials.

Piercing system 1300 also may include a top 1354 positioned at second end 1306. Top 1354 may include a base 1355 positioned over septum 1314 and the opening of cartridge 1302. Top 1354 may include a chamber 1356 extending from base 1355 in a direction away from piston 1316. Chamber 1356 defines a cavity 1357 and includes an opening 1358 in communication with cavity 1357. In some embodiments, top 1354 may be integrated with septum 1314 (e.g., integral or of one-piece construction). In alternative embodiments (not shown), top 1354 may be provided or initially assembled on fluid conduit 300 and not installed directly on/with cartridge 1302 and/or integrated with septum 1314.

A portion of fluid conduit 300, such as needle 308, a tube or the like, may extend through opening 1358 of chamber 1356 and into cavity 1357, but not through base 1355 in the pre-activated state. Opening 1358 may be pre-formed, or may be formed by the penetration of needle 308 through chamber 1356. Opening 1358 of chamber 1356 may form a sterile sliding seal about needle 308 such that pathogens or other contaminants are prevented from passing into cavity 1357. Needle 308 can move relative to top 1354 without disrupting the sterile seal therebetween. Cavity 1357 may be sterile or aseptic such that the inner surfaces of cavity 1357 and needle 308 are sterile. In another embodiment, cavity 1357 may be sterilized after needle 308 is inserted through opening 1358 and into cavity 1357. In alternative embodiments, rather than top 1354, a convoluted flexible (e.g., rubber) bellows or bladder member may form cavity 1357 and allow translation of cartridge 1302 relative to needle 308 (or vice versa). The flexible member also may seal or form cavity 1354 about needle 308 after sterilization.

Piston 1316 may be coupled to a translation mechanism 1366 that is configured to translate piston 1316 and cartridge 1302 in a direction toward second end 1306. The movement of piston 1316 toward second end 1306 causes piston 1316 to act against the contents within cartridge 1302 (e.g., drugs, medications), which ultimately transfers force against second end 1306 of cartridge 1302, causing cartridge 1302 to move along longitudinal axis 40. Translation mechanism 1366 may include a 12 mm motor with a five-stage gear reduction (360:1). Translation mechanism 1366 may have spring contacts that create an electrical connection with an associated printed circuit board (e.g., first electronic board 1402). The motor may be configured to generate a torque of about 136 mN*m at 36 rpm. These design parameters of the motor are merely exemplary, and any other suitable motor also may be utilized.

Translation mechanism 1366 may include a leadscrew mechanism coupled to piston 1316 that extends axially upon relative rotation about longitudinal axis 40. This telescoping leadscrew may have a 100 N output, a 20 mm stroke, and a 7°/45° buttress thread shape with a 0.75 mm pitch. The materials for the leadscrew mechanism may include acetal and polybutylene terephthalate. The leadscrew mechanism may extend within piston 1316 to reduce dead space behind piston 1316. While piston 1316 is shown in FIGS. 13 and 14 with longitudinally spaced threads, in some examples, such threads may not be present. In another exemplary embodiment (not shown), translation mechanism 1366 may include a manually engageable surface or member that is manually manipulated by a user to move piston 1316. For example, piercing system 1300 may include a cartridge or a plunger coupled to the back side of piston 1316. In another exemplary embodiment (not shown), translation mechanism 1366 may include a pneumatic or hydraulic drive member that is actuated or initiated by a user to move piston 1316. The drive member may be in the form of expanding bellows, an expanding bladder, an expanding diaphragm, or a sliding seal or piston, for example. The direct pneumatic or hydraulic pressure may provide the force required to move piston 1316.

Piercing system 1300 also includes a collar 1390 coupled or fixed to second end 1306. Collar 1390 may include a plurality of circumferentially spaced apart fingers 1392 that engage and surround neck 1310. Collar 1390 may be fixed, or otherwise coupled to second end 1306. Collar 1390 may include a wall 1390a that extends at least partially about neck 1310, the opening of second end 1306, cap 1312, septum 1314, and/or top 1354. Wall 1390a of collar 1390 may be positioned radially or laterally outward of neck 1310 and extend longitudinally past neck 1310, cap 1312, and septum 1314.

In the pre-activated state of piercing system 1300 shown in FIG. 13, an edge 1393 of collar 1390 may engage a corresponding radially or laterally inwardly extending cam, latch or actuation portion 1394 of a driver retainer member 1395. Retainer member 1395 may be slidable relative to collar 1390. Collar 1390 and retainer member 1395 may be configured such that in the pre-activated state or arrangement shown in FIG. 13, at least a portion of the cam or actuation portion 1394 of retainer member 1395 is positioned directly behind a retaining portion 1399 of a driver 1398 slidable within retainer member 1395. A wall 1391 of driver 1398 may extend into and through an end cap portion 1396 of retainer member 1395 and into an interior portion of retainer member 1395, and retaining portion 1399 of driver 1398 may extend radially outward from wall 1391. In some embodiments, wall 1391 of driver 1398 may be substantially cylindrical and retaining portion 1399 of driver 1398 may be a flange extending about an end of the wall 1391.

In the pre-activated state of piercing system 1300, an elastically deformed biasing or resilient member 1397 may be positioned between cap portion 1396 of retainer member 1395 and retaining portion 1399 of driver 1398. Biasing member 1397 may exert a force against driver 1398 in the pre-activated state of piercing system 1300 acting in the direction towards cartridge 1302. Biasing member 1397 may be any member effective in applying the force in the pre-activated state, and then releasing such force upon activation, as discussed below with reference to FIG. 14. In some embodiments, biasing member 1397 may be a conical or flat spring.

Needle 308 of fluid conduit 300 may be fixed or coupled to driver 1398 such that fluid conduit 300 moves with driver 1398. In the pre-activated state of piercing system 1300, needle 308 may be positioned within the sterile cavity 1357, but not through base 1355 of top 1354, septum 1314, and/or into cavity 1308 of cartridge 1302.

In some embodiments, in lieu of cavity 1357, needle 308 may be positioned within a plug when the piercing system 1300 is the pre-activated state. The plug may be a solid plug which is devoid of any holes, cavities, or openings, and which may be formed of a first rubber material. The first rubber material may be permeable to a sterilizing gas, such as, e.g., ethylene oxide or vaporized hydrogen peroxide. The first rubber material may include one or more of isoprene, ethylene propylene diene monomer (M-class) rubber (EPDM), and styrene-butadiene, among others. The permeability of the first rubber material to a sterilizing gas may allow needle 308, when disposed within the plug, to be sterilized before use. The plug may be molded about needle 308, so that needle 308 is impaled into the plug.

To move piercing system 1300 from the pre-activated state of FIG. 13, translation mechanism 1366 may be activated to move piston 1316 towards second end 1306 and translate cartridge 1302 along longitudinal axis 40 toward driver 1398. Because the needle 308 is not yet in fluid communication with cartridge 1302, activation of translation mechanism 1366 applies a pressure against the fluid contained in cartridge 1302, which is then applied to cartridge 1302 itself. This pressure also causes edge 1393 to push against and deflect actuation portion 1394 radially outward. Without actuation portion 1394 blocking its path, retaining portion 1399 and needle 308 are moved toward cartridge 1302 by the expansion of biasing member 1397. Driver 1398 may be coupled to flange 204 of carrier 202, and thus, this movement of driver 1398 toward cartridge 1302 also may move carrier 202 in the same direction. This movement corresponds to the movement of carrier 202 relative to housing 3 in FIGS. 6 and 7, which enables protrusion 330 to clear impediment 600 to inject needle 306.

The movement of needle 308 toward second end 1306 of cartridge 1302 also causes needle 308 to pierce through base 1355 of top 1354, septum 1314, and cavity 1308, into fluid communication with the contents of cartridge 1302. Once needle 308 is in fluid communication with cartridge 1302, further movement of piston 1316 toward second end 1306 urges fluid through needle 308 and a remainder of fluid conduit 300. In some embodiments, piercing system 1300 may be configured such that, after activation, no more of needle 308 than the portion that was already positioned within sterile cavity 1357 extends into cavity 1308. This may help prevent contamination of the contents of cartridge 1302 with non-sterile portions of needle 308.

Biasing member 1397 may be configured to expand such that fluid conduit 300 pierces top 1354 and/or septum 1314 at a high speed, such as at a speed of at least about 10 mm/sec, or at least about 40 mm/sec. The relatively quick piercing of top 1354 and/or septum 1314 via biasing member 1397 may help prevent leakage of the contents of cavity 1308 which may be under pressure via piston 1316.

After drugs have been delivered to the user via needle 306, needle 306 may be automatically withdrawn from the user. Referring to FIGS. 12-14, translation mechanism 1366 may be operated in a reverse mode such that the rotation of the lead screw is in an opposite direction compared to the insertion step. This counter-rotation may cause piston 316 to move back toward first end 1304, and also cause cartridge 1302 to move in an opposite direction along axis 40 (as compared to during fluid delivery and insertion of needle 306). The movement of cartridge 1302 in the opposing direction may cause ramp 1500 in FIG. 12 (which is attached to wall 1391) to push against ramp 243 of stop 240. This may cause stop 240 to deflect about its fixed end 241 in the direction of arrow 240a, and allow shuttle 340 to move from its second position to its third position to retract needle 306 as set forth above. In this way, withdrawal and insertion of the needle into a patient can both be accomplished with a single spring within the device.

It is further contemplated that fluid conduit 300 may be the only fluid conduit of auto-injector 2 configured to be in fluid communication with cartridge 1302. Thus, drugs from cartridge 1302 may be deployed only through fluid conduit 300 and into the user during normal operation of auto-injector 2. Additionally, needle 306 may be the only needle of auto-injector 2 configured to be deployed into a patient. In this way, a single piece of metal or plastic can be used to carry the fluid from cartridge 1302 to a patient.

Locking Component (Drop Pin)

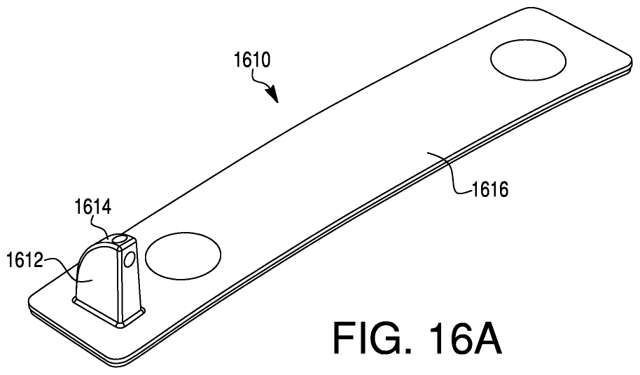

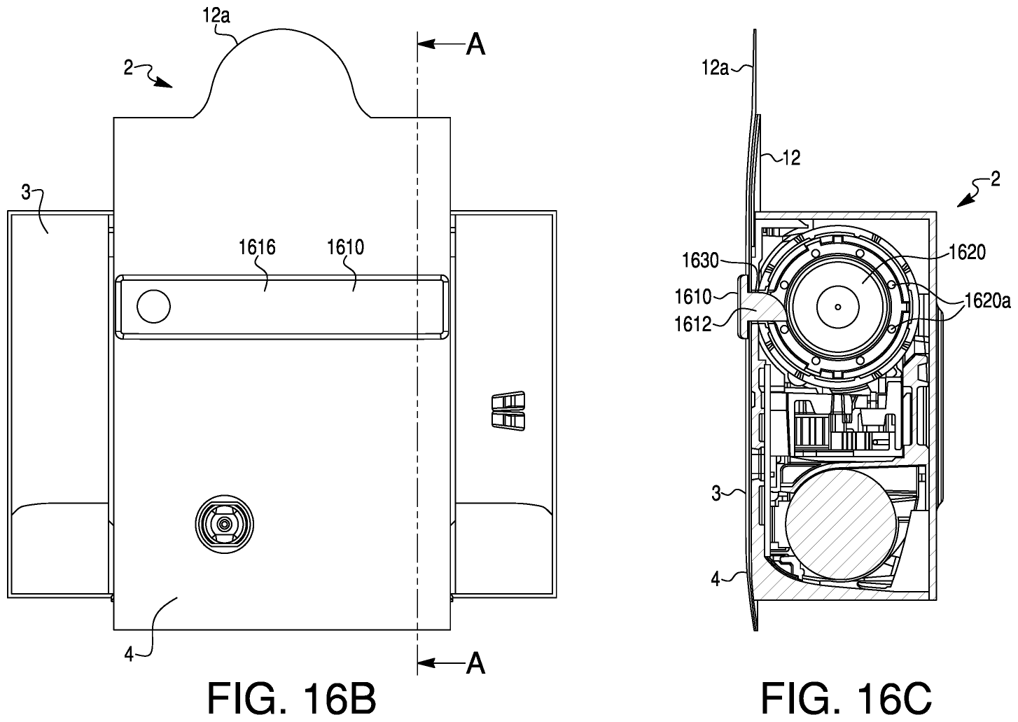

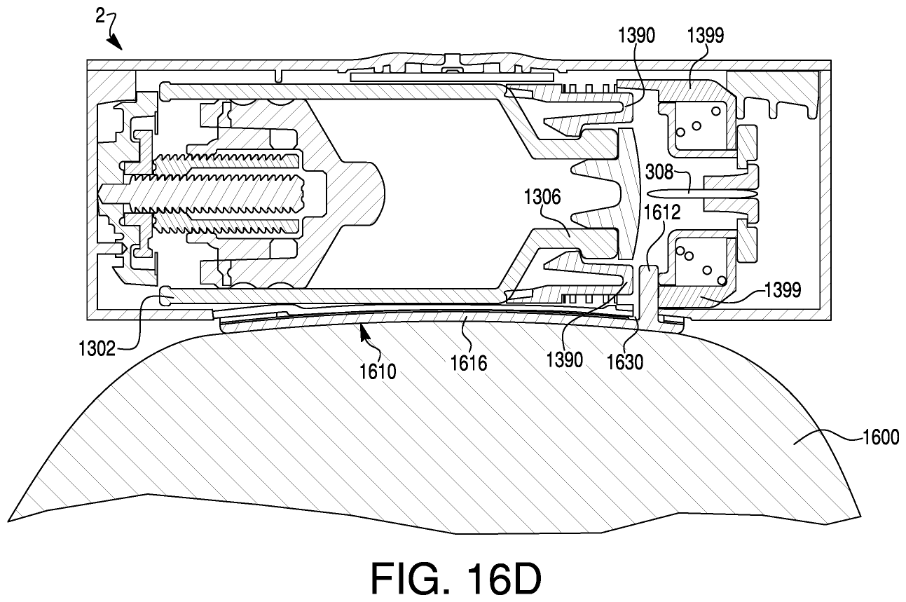

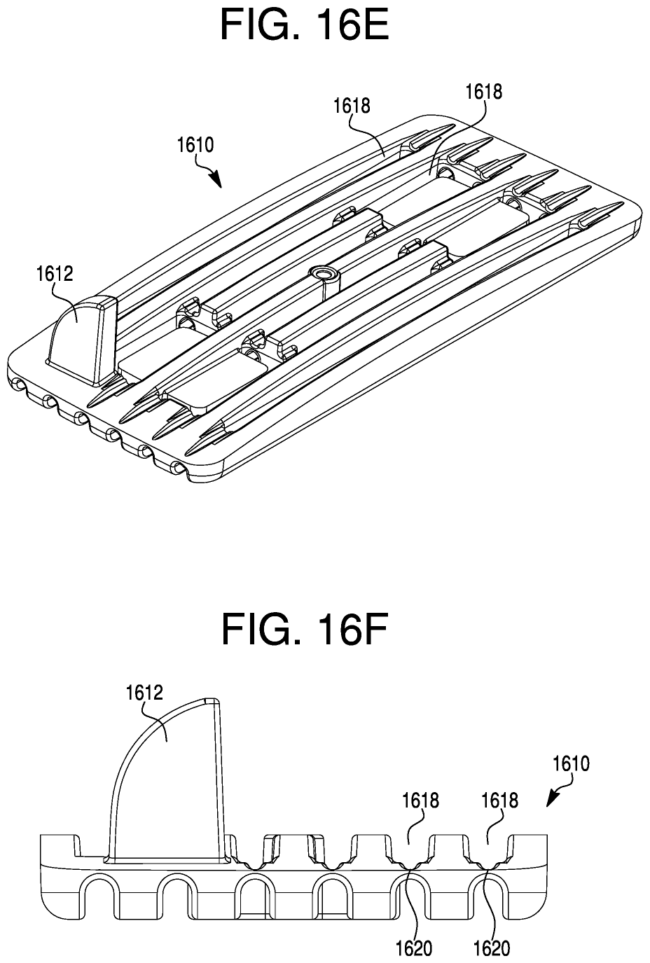

Referring to FIGS. 16A-16D, auto-injector 2 may include a locking component 1610. As shown in FIG. 16A, locking component 1610 may include a lock (e.g., a protrusion) 1612 having a curved surface 1614 and may further include a cover portion 1616. Cover portion 1616 may be shaped to conform to tissue-engaging surface 4 of auto-injector 2, as shown in FIG. 16D. Cover portion 1616 and tissue-engaging surface 4 may be concave to receive an anatomical portion 1600 of a user. Anatomical portion 1600 may be, for example, a thigh, hip, arm, posterior, or any other area of the body suitable for injection. Lock 1612 may be connected to cover portion 1616. In some embodiments, locking component 1610 may be formed as a single piece, such that lock 1612 and cover portion 1616 are integrally connected. Locking component 1610 may be formed from any suitable rigid or semi-rigid material. Locking component 1610 may, for example, be formed of acrylonitrile butadiene styrene (ABS) and may further have a frosted clear appearance indicating that locking component 1610 is disposable.

As shown in FIGS. 16B-16C, locking component 1610 may be disposed on or adjacent tissue-engaging surface 4 of the auto-injector 2 such that lock 1612 may extend into auto-injector 2. Locking component 1610 may further be disposed on or adjacent a liner 12a which may initially cover adhesive patch 12 prior to use of auto-injector 2. Locking component 1610 may be positioned relative to liner 12a such that upon removal of liner 12a from adhesive patch 12, locking component 1610 may also be removed from tissue-engaging surface 4. Lock 1612 may extend into auto-injector 2 via lock opening 1630 formed in tissue-engaging surface 4. When locking component 1610 is disposed on or adjacent tissue-engaging surface 4, cover portion 1616 may be attached to tissue-engaging surface 4 via an adhesive disposed between cover portion 1616 and tissue-engaging surface 4. Locking component 1610 may be disposed on or adjacent tissue-engaging surface 4 such that it is selectively removable by a user.

Referring to FIGS. 16C-D, when locking component 1610 is disposed on or adjacent tissue-engaging surface 4, lock 1612 may extend into auto-injector 2 such that it prevents movement of one or more internal mechanisms of auto-injector 2. For example, when locking component 1610 is disposed on or adjacent auto-injector 2, lock 1612 may extend into auto-injector 2 such that lock 1612 engages with one or more internal components of auto-injector 2, preventing those components from moving and/or being activated.

Referring to FIG. 16D, when locking component 1610 is disposed on or adjacent tissue-engaging surface 4, lock 1612 may extend into auto-injector 2 such that it is disposed within piercing system 1300. As described herein previously, collar 1390 may be coupled or fixed to second end 1306 of cartridge 1302. As also described herein previously, when piercing system 1300 is moved from the pre-activated state, cartridge 1302 and consequently collar 1390 may translate in a direction parallel to a longitudinal axis of cartridge 1302 toward retaining portion 1399. When lock 1612 extends into auto-injector 2 and is adjacent to collar 1390, lock 1612 may prevent cartridge 1302 from translating toward retaining portion 1399 or otherwise prevent cartridge 1302 and collar 1390 from applying a force against actuation portion 1394. Thus, even if the motor were somehow activated while lock 1612 is disposed in its locking position, fluid communication between needle 308 and cartridge 1302 could not be established and needle 306 could not be deployed outside of housing 3. Furthermore, when in the locking position, lock 1612 may prevent the movement of cartridge 1302 toward needle 308, thereby preventing deflection of actuation portion 1394 and consequently preventing retaining portion 1399 and needle 308 from moving toward cartridge 1302. In the event of auto-injector 2 being dropped or being subject to vibrations, lock 1612 may further prevent piercing system 1300 from being moved from the pre-activated state and consequently may prevent cartridge 1302 from being pierced by the needle 308.

When locking component 1610 is disposed on or adjacent tissue-engaging surface 4, locking component 1610 may additionally serve as a spacer between a user's skin and tissue-engaging surface 4. For example, locking component 1610 may have a thickness such that touch sensor 1410, described in greater detail hereinafter, is unable to detect the user's skin thereby avoiding inadvertent activation of auto-injector 2. Locking component 1610 may have a thickness, for example, from about 1 mm and about 5 mm, or about 3 mm.

Accordingly, locking component 1610 may act as an effective safety mechanism to prevent inadvertent activation of auto-injector 2. When locking component 1610 is disposed on or adjacent the tissue-engaging surface 4, lock 1612 may prevent various internal components of auto-injector 2 from moving. In the event auto-injector 2 is dropped on the floor prior to use, for example, locking component 1610 may prevent inadvertent piercing of cartridge 1302 and/or inadvertent initiation of an injection sequence. Locking component 1610 may also prevent such movement and/or inadvertent initiation of an injection sequence should auto-injector 2 be subjected to vibration during transport.

If a user wishes to use and/or is ready to use auto-injector 2, the user may separate locking component 1610 from tissue-engaging surface 4, thereby removing lock 1612 from lock opening 1630. The user may, for example, peel cover portion 1616 off of tissue-engaging surface 4. Alternatively, the user may peel liner 12a away from adhesive patch 12, thereby removing locking component 1610 from tissue-engaging surface 4. When separating locking component 1610 from tissue-engaging surface 4, curved surface 1614 may allow lock 1612 to rock within lock opening 1630, thereby allowing lock 1612 to be easily removed from lock opening 1630. With lock 1612 removed from lock opening 1630, auto-injector 2 may be in a state in which it is ready to be used such that, e.g., an injection sequence may be initiated.

FIGS. 16E and 16F depict locking component 1610 according to some embodiments. As shown in FIGS. 16E and 16F, locking component 1610 may have an increased width (relative to the depiction of locking component 1610 in FIGS. 16A-16C) to ensure locking component 1610 extends over touch sensor 1410 when locking component 1610 is disposed on auto-injector 2. Moreover, locking component 1610 may have a ribbed structure and may include air gaps or recesses 1618 and hinges 1620. Air gaps 1618 may inhibit conduction of a capacitive field between the user's skin and touch sensor 1410 when the auto-injector 2 is placed near the user with locking component 1610 in place. Hinges 1620 may allow locking component 1610 to flex when locking component 1610 is peeled from auto-injector 2. In some embodiments, a removable cover other than and/or separate from locking component 1610 may extend over touch sensor 1410 to inhibit inadvertent skin detection.

Electronics

FIG. 4A shows a control system 1400 of auto-injector 2. Control system 1400 may include components positioned on a first electronics board 1402 and a second electronics board 1404, and also may include a power source 1406. First electronics board 1402 may include a controller 1408, an activating switch 1409, a touch sensor 1410, a needle insert switch 1412, and an emitter 1414. Second electronics board 1404 may include a detector 1416, an audio module 1418, a visual module 1420, and a haptic module 1422. Though FIG. 4A depicts audio module 1418, visual module 1420, and haptic module 1422 as included in second electronics board 1404, in some embodiments, one or more of the foregoing modules may be included on the first electronics board 1402 One or more of the components of first electronics board 1402 and second electronics board 1404 may be operatively coupled to controller 1408, and powered by power source 1406. Controller 1408 also may be operatively coupled to translation mechanism 1366, and may be configured to control operation of translation mechanism 1366 to initiate and control needle insertion and retraction as set forth above. Translation mechanism 1366 may be coupled to first electronics board 1402 via one or more spring contacts during a final assembly step where cartridge 1302 is inserted into housing 3. As described herein previously, translation mechanism 1366 may include a motor, gearing, and a leadscrew mechanism.

The majority of the assembly of auto-injector 2 may occur, e.g., on an assembly line at a manufacturing facility. Then, two device halves (or portions) may be shipped to a drug filling or final assembly facility. Indeed, the two separate portions 1490 and 1492 need not be the same size, as illustrated in FIG. 4B. Once a drug vial, e.g., cartridge 1302, is filled with a drug or other medicament, cartridge 1302 may be assembled with a remainder of auto-injector 2. For example, the two device halves (portions 1490 and 1492) may be assembled together with the filled drug cartridge 1302 therein. In one example, portion 1490 and translation mechanism 1366 may be snapped in place behind cartridge 1302. Portion 1490 may be part of housing 3 including a base or module configured to contain translation mechanism 1366 and its associated electronics. Portion 1492 may be a part of housing 3 containing substantially all of the other components described herein, including, e.g., the needle mechanism, sterile connector, and piercing mechanisms described herein. In this example, an electrical connection of the motor of translation mechanism 1366 must be made during the snapping of translation mechanism 1366 behind cartridge 1302 (i.e., during the assembly step where portions 1490 and 1492, and cartridge 1302 are combined to form a complete and functional auto-injector 2). To accommodate such an electrical connection, the drivetrain of translation mechanism 1366 may include one or more spring contacts 1494 (referring to FIG. 4C) that will contact pads 1495 (also referring to FIG. 4C) on the first electronics board 1402 upon assembly. Though not depicted in FIG. 4C, the drivetrain of translation mechanism 1366 may include additional spring contacts that may contact additional pads on the first electronic board 1402 upon assembly. Such additional spring contacts and additional pads may serve to connect additional components of the translation mechanism 1366, such as a tachometer, a motor encoder, or any other sensors or devices, to the first electronics board 1402. Thus, the connection of translation mechanism 1366 to first electronics board 1402 (including controller 1408) may be made without any loose wires or other similar structures.

Such an assembly process may be relatively simpler than simpler devices (e.g., auto-injectors) with relatively more complex final assembly processes. As a result, the contemplated assembly process described herein may lead to a reduction of labor costs.

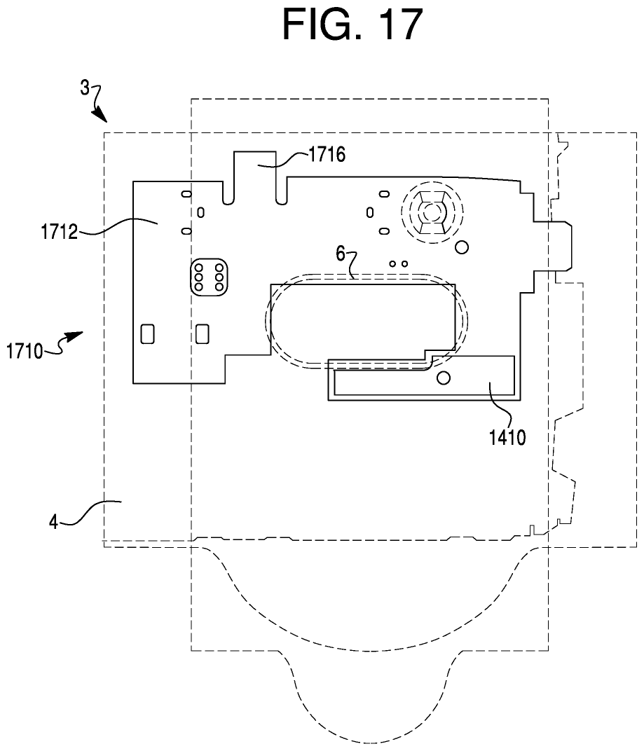

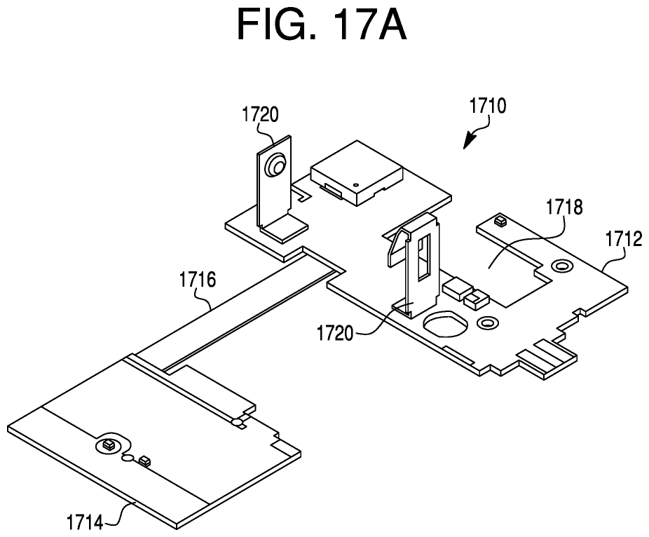

In some embodiments, auto-injector 2 may include a single (i.e., only or exactly one) electronics board 1710 as shown in FIGS. 17 and 17A, on which the components of control system 1400 described herein previously may be positioned. As shown in FIG. 17A, electronics board 1710 may include a first board segment 1712 and a second board segment 1714. The first board segment 1712 and the second board segment 1714 may be physically and electrically connected via a flexible segment 1716. Flexible segment 1716 may be, for example, a ribbon cable, a flexible conductive substrate, or the like. In some embodiments, flexible segment 1716 may be formed of fiberglass board that is machined thinly enough to flex, and is sometimes referred to as “semi-flex.” In some embodiments, flexible segment 1716 may be formed of a flexible polymer. The flexible polymer may be formed by a process sometimes referred to as “rigid-flex” in which a sandwich of a first portion fiberglass, flexible polymer, and second portion of fiberglass is first formed. The first and second portions of fiberglass may be subsequently removed to leave the thin flexible polymer portion.

Electronics board 1710 may include one or more brackets 1720 for mounting or otherwise securing electronics board 1710 to an interior of auto-injector 2. The first board segment 1712 may further include a cutout 1718. The cutout 1718 may be positioned such that first board segment 1712 may be positioned to allow the needle to pass through cutout 1718 when deployed.

In some embodiments, first board segment 1712 may correspond to first electronics board 1402 and second board segment 1714 may similarly correspond to second electronics board 1404, each as described herein previously. By connecting first board segment 1712 and second board segment 1714 via flexible segment 1716, first board segment 1712 may be positioned adjacent to tissue-engaging surface 4 of the auto-injector 2 whereas second board segment 1714 may be positioned on an opposite side of auto-injector 2 toward upper portion 30 of housing 3. Accordingly, the single electronics board 1710 may be utilized to both connect components located toward tissue-engaging surface 4 and connect components located toward upper portion 30. Such a configuration may allow for ease of assembly of the auto-injector 2 by obviating a need for complex wiring or soldering.

As shown in FIG. 17, electronics board 1710 may be positioned within housing 3. First board segment 1712 may be positioned adjacent to tissue-engaging surface 4 whereas second board segment 1714 may be positioned on an opposite side of auto-injector 2 (e.g. behind first board segment 1712 in FIG. 17). Flexible segment 1716 may be flexed or folded to maintain a connection between first board segment 1712 and second board segment 1714 in such positions.

Touch sensor 1410 may be incorporated in or on first board segment 1712 of electronics board 1710. To allow for adequate detection of a user's skin, touch sensor 1410 and first board segment 1712 may be located close to tissue-engaging surface 4 of housing 3. Tissue-engaging surface 4 of housing 3, or a portion thereof adjacent to touch sensor 1410, may be sufficiently thin such that an electric field of detectable magnitude may form between touch sensor 1410 and a user's skin. In some embodiments, the portion of tissue-engaging surface 4 adjacent touch sensor 1410 may be less than about 2 mm, about 1 mm, or less than about 1 mm. Further, the portion of tissue-engaging surface 4 adjacent touch sensor 1410 may be made from a solid material, such as plastic. By forming the portion of tissue-engaging surface 4 adjacent touch sensor 1410 from a solid material, as opposed to a ribbed, cored, or hollow material, a dielectric constant between the user's skin and touch sensor 1410 may optimize a responsiveness of touch sensor 1410.

Additionally, touch sensor 1410 may be positioned in or on electronics board 1710 so as to be adjacent to or near opening 6 through which the needle may be deployed. By positioning touch sensor 1410 adjacent to or near opening 6, a likelihood that touch sensor 1410 may detect a user's skin when auto-injector is positioned appropriately is increased. Furthermore, a curvature of tissue-engaging surface 4 may decrease the likelihood that touch sensor 1410 may falsely interpret a flat surface such as a tabletop to be a user's skin by creating a space between touch sensor 1410 and the flat surface.

By incorporating touch sensor 1410 in or on electronics board 1710, a need for one or more wires and/or other circuitry connecting touch sensor 1410 to a separate electronics board may be eliminated. Assembly of the auto-injector 2 may thereby be simplified and a cost of the auto-injector may be reduced.

As electronics board 1710 may be located adjacent to tissue-engaging surface 4, electronics board 1710 may include a cutout to allow the needle to be deployed through electronics board 1710 and subsequently through opening 6. Further, electronics board 1710 may be positioned such that touch sensor 1410 is directly adjacent opening 6 and no gap exists between an edge of touch sensor 1410 and opening 6. Alternatively, electronics board 1710 may be positioned such that a gap exists between an edge of touch sensor 1410 and opening 6 and the gap has a maximum of width of 5 mm, 2 mm, or 1 mm, for example.

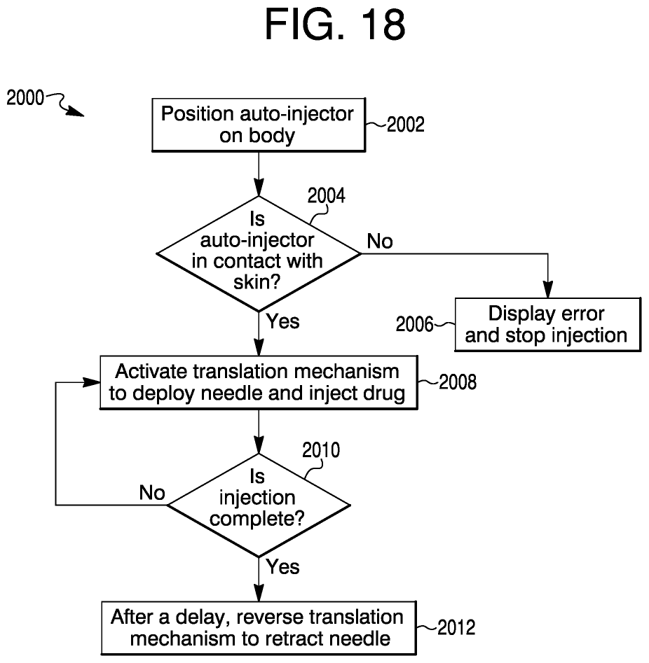

Controller 1408 may be configured to accept information from the system and system components described above, and process the information according to various algorithms to produce control signals for controlling internal mechanisms of auto-injector 2, including translation mechanism 1366. Examples of such algorithms are described hereinafter with reference to FIGS. 18 and 20-23. The processor may accept information from the system and system components, process the information according to various algorithms, and produce information signals that may be directed to audio module 1418, visual module 1420, haptic module 1422, or other indicators of, e.g., second electronics board 1404, in order to inform a user of the system status, component status, procedure status or any other useful information that is being monitored by the system. The processor may be a digital IC processor, analog processor or any other suitable logic or control system that carries out the control algorithms.

As discussed above with respect to FIGS. 3A and 3B, activating switch 1409 may be a mechanical plunger-type switch that extends away from tissue-engaging surface 4 of auto-injector 2. Activating switch 1409 may include an electrical circuit that is complete unless activating switch 1409 is depressed. For example, when auto-injector 2 is attached to a user's skin, switch 1409 may be depressed, breaking the electrical circuit, and indicating to controller 1408 that auto-injector 2 should be activated. In order to conserve power, the components of auto-injector 2 may be in an idle or sleep mode until switch 1409 is activated. In yet another example, auto-injector 2 may not be powered at all until switch 1409 is activated, and deactivation of switch 1409 may cut off power to auto-injector 2 entirely. While a mechanical plunger-type switch is disclosed, any other suitable mechanism for activating auto-injector 2 may be utilized, including, e.g., a button depressed by the user, voice signals, and a wireless signal from another electronic device, among others.

Touch sensor 1410 may be configured to help controller 1408 determine whether auto-injector 2 is properly deployed on the skin of a user. In one example, touch sensor 1410 may be a capacitive sensing electrode or any other device configured to differentiate contact with skin versus other materials, such as, e.g., wood, plastic, metal, or another material. When skin is in the proximity of the capacitive sensing electrode, a signal indicative of such contact may be sent to controller 1408. Thus, touch sensor 1410 may serve to verify that auto-injector 2 is properly placed on a user's skin, even if switch 1409 is depressed. Touch sensor 1410 may include a capacitive sensing electrode coupled to first electronics board 1402 and also to an interior of housing 3. Housing 3 and adhesive patch 12 may act as an overlay (insulator) that acts as a dielectric between the skin of the user and the capacitive sensing electrode. Alternatively, touch sensor 1410 may be incorporated in or on electronics board 1710, as described herein previously, such that the capacitive sensing electrode is also incorporated in or on electronics board 1710, Contact of portions of housing 3 and/or adhesive patch 12 near the capacitive sensing electrode may cause the capacitance of the electrode to increase, for example, by about 1 to about 10 pF, indicating placement of auto-injector 2 on a skin surface.

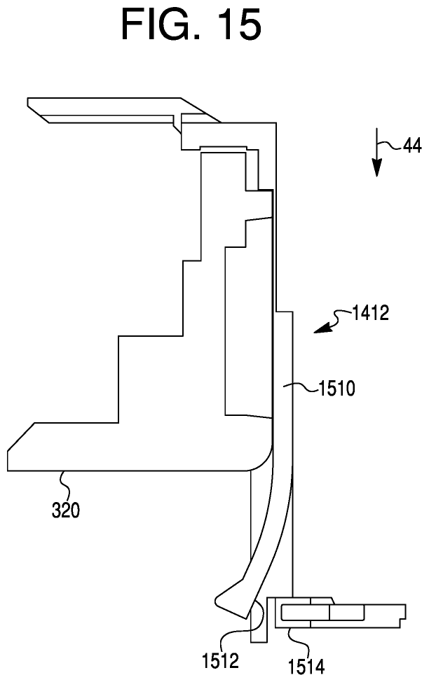

Needle insert switch 1412 may be configured to send a signal to controller 1408 that needle 306 is deployed within a user. For example, referring to FIG. 15, needle insert switch 1412 may include a curved cantilever 1510 including a first contact 1512. Needle insert switch 1412 also may include a second contact 1514. First contact 1512 may be placed into electrical contact with second contact 1514 when needle 306 is deployed into the user. During deployment of needle 306, driver 320 may move downward along axis 44 and deflect curved cantilever 1510 and first contact 1512 toward second contact 1514. When first contact 1512 and second contact 1514 connect to one another, a signal may be sent to controller 1408 indicating that needle 306 has been successfully deployed into the user. The separation of first contact 1512 and second contact 1514 may indicate that needle 306 has been retracted from the user.

Emitter 1414 and detector 1416 may operate as an optical interruption sensor, or photo-interrupter in order to allow controller 1408 to determine a state of auto-injector 2. Emitter 1414 may be a light emitting diode (LED) or other suitable light emitter, and detector 1416 may be, e.g., a phototransistor configured to receive light emitted by emitter 1414. In one example, emitter 1414 may emit infrared light, although other suitable wavelengths of light also may be used. The use of infrared light may help reduce interference from external light.

As shown in FIG. 13B, emitter 1414 and detector 1416 may be arranged across from one another within housing 3 to enable a beam of light 1430 to pass from emitter 1414, through cartridge 1302, to detector 1416. Cartridge 1302, and any fluid contained therein may be at least partially transparent to beam 1430 so that beam 1430 may pass through cartridge 1302 and its contents. As piston 1316 is moved toward second end 1306 during drug delivery (referring to FIGS. 13 and 14), piston 1316, and in particular a shoulder of piston 1316, may interrupt beam 1430. When detector 1416 fails to sense beam 1430, a signal may be sent to controller 1408, which may interpret the signal to indicate an end of an injection (e.g., that all of the drug contained within cartridge 1302 has been expelled). In some examples, the refraction path of beam 1430 may be considered when positioning emitter 1414 and detector 1416 relative to one another. For example, beam 1430 may be refracted as it passes through cartridge 1302 and any liquid contained therein, and emitter 1414 and detector 1416 may be offset from one another accordingly. Additionally, emitter 1414 and detector 1416 may be offset from a center of housing 3 so that the shoulder of piston 1316 may block beam 1430. In at least some examples, an optical interruption sensor or similar mechanism may help avoid false positives in the event of a drive train failure. That is, the optical switch may help controller 1408 determine that an injection was not completed with greater accuracy than other mechanisms.

Audio module 1418 may include a speaker or the like to provide audio feedback to the user. Openings in housing 3 may facilitate the travel of sound from audio module 1418 to the user. Audio module 1418 may generate a tone or other sound at the start and at the end of injection, and/or to indicate any other benchmark during the injection, such as an error, for example. Visual module 1420 may include one or more LEDs or similar devices to provide visual feedback to the user. Visual module 1420 may include different colored LEDs to provide various messages to the user. For example, a plurality of blue LEDs arranged in a ring could be used to display progress of the injection over time, one or more green LEDs could be used to display completion of the injection, and a red LED could be used to display an error to the user. Any other suitable colors, combinations, and/or numbers of LEDs may be used in various examples. For example, a combination of red, blue, and purple LEDs may be utilized. In one arrangement, eight LEDs may be arranged in a circle having a diameter of about 26.5 mm, or a diameter from about 10.0 mm to about 40.0 mm. It is to be understood that this exemplary quantity and positioning of LEDs is not intended to be limiting and any quantity and/or positioning of LEDs may be used. The LEDs may be activated sequentially around the circle to indicate progress of an injection (e.g., in a progress ring arranged in a similar manner as a clock—see, for example, LEDs 52 on FIG. 4B). Controller 1408 also may be configured to receive feedback from various sensors, and rescale a speed that various LEDs are activated based on feedback from the sensors. For example, the LEDs in the progress ring may be activated in three or more operation phases including, e.g., an injection sequence activation phase, an injection phase, and a retraction phase. Those of ordinary skill in the art will recognize that auto-injector 2 may have more or less than the above-described three operation phases. There may be an expected time for completing each phase, but there also may be some variability in the actual times experienced during any of the aforementioned operation phases of auto-injector 2. An algorithm may be utilized to help avoid the premature activation of LEDs, for example, when a certain phase finishes earlier than expected, or to have progress along the ring stopped when a certain phase takes longer than expected. At any given point, the algorithm may divide the remaining estimated time for completion of drug delivery by the number of unactivated LEDs in the progress ring, to determine a rate at which the remaining LEDs in the progress ring should be activated.

For example, before the injection sequence activation phase, the LEDs may be activated at a rate equal to the estimated time of the entire drug delivery process (e.g., the estimated time to complete all of injection sequence activation phase, the injection phase, and the retraction phase) divided by the total number of unactivated LEDs in the progress ring. Stated differently, the estimated time of the entire drug delivery process may be divided by a number that is the total number of LEDs in the progress ring less any already-activated LEDs. Thus, if, for example, one LED is already activated, the estimated time of the entire drug delivery process may be divided by one less than the total number of LEDs in the progress ring.

After completion of the injection sequence activation phase, the LEDs may be activated at a rate equal to the sum of estimated times for completing the remaining phases (e.g., the injection phase and the retraction phase) divided by the number of unlit LEDs in the progress ring. After completion of the injection phase, the LEDs may be activated at a rate equal to the estimated time to complete the retraction phase, divided by the number of unlit LEDs.

In some embodiments, subsets of LEDs may be used to indicate progress of injection phases. For example, in embodiments having eight LEDs positioned on a housing of auto-injector 2, a first LED may be illuminated to indicate needle insertion. The second through seventh LEDs may then be illuminated sequentially to indicate a progress of the injection phase. Lastly, the eighth LED may be illuminated to indicate needle retraction. While an exemplary configuration of the LEDs and corresponding logic has been described, it should be understood that the quantities of LEDs for each phase of an injection process may be varied as desired.

Visual module 1420 also may include a display screen, touch screen, or other suitable device to provide one-way or two-way communication with the user. Visual module 1420 may be visible by the user from outside of housing 3 via a window in housing 3. Haptic module 1422 may include, e.g., a haptic motor configured to generate vibrations that can be felt by the user. Vibrations may signal the start and the end of an injection, and/or may help provide additional information to a user.

Controller 1408 may be coupled to a wireless communication module and an antenna. The wireless communication module may be configured to transmit data from controller 1408 to, e.g., a mobile device, computer, cell phone, or the like. The wireless communication module may be configured to transmit information over one or more wireless modalities, such as, e.g., Bluetooth, Bluetooth low energy (BLE), near-field communication (NFC), infrared, cellular networks, and wireless networks, among others. The antenna may be any suitable device configured to assist the wireless communication module in data transmission and/or amplification. Thus, controller 1408 may be configured to transmit diagnostic information of the user and/or auto-injector 2, information pertaining to completion of an injection, and/or information pertaining to an error state of auto-injector 2 to a device of the user, or to the cloud. Signals indicative of needle insertion and/or early device removal also could be transmitted via the wireless communication module. Controller 1408 may also be configured to transmit temperature information for auto-injector 2. For example, a user may be able to monitor, via a mobile device and/or application, for example, a temperature of auto-injector 2 when auto-injector 2 is removed from refrigeration. Controller 1408 also may receive activation and/or delay commands via the wireless communication module. Controller 1408 may further receive operation adjustment commands such as commands relating to adjustment of preferred operation speed, for example. In some embodiments, controller 1408 may receive a command to pause an injection.

In some embodiments, controller 1408 may communicate with a mobile application of a user's mobile device via the wireless communication module. The mobile application may be configured to facilitate use of auto-injector 2 and improve user experience. In some embodiments, the mobile application may be used to automatically check an expiration date of a medicament contained within auto-injector 2. Such functionality may relieve a user from having to manually check the expiration date and may improve user safety. Based on an expiration date, the mobile device may be configured to alert the user and/or disable use of the auto-injector 2. In some embodiments, the mobile application may be used to alert a user as to product recalls and/or may disable the device in the event of product recalls. For example, the mobile application may access a database via the internet to determine whether particular devices, lots of devices, medicaments, and/or lots of medicaments have been recalled. In some embodiments, the mobile application may be configured to confirm whether the auto-injector 2 and/or medicament is authentic as opposed to counterfeit. The mobile application may do so by, for example, cross-referencing a product serial number or a digital signature against a database of authenticated products. In some embodiments, a portion or portions of auto-injector 2 may be disposable and the mobile application may be configured to confirm the authenticity of such portion or portions prior to use.

In some embodiments, the mobile application may be used to facilitate an injection sequence. For example, the mobile application may sync with the events of an injection sequence and provide contemporaneous instructions to the user as to which tasks (e.g., depress switch 1409, hold auto-injector 2 against skin, remove auto-injector) to perform at which times. In some embodiments, the instructions may be narrated audibly. In some embodiments, the instructions may be provided visually via a display on the mobile device. In some embodiments, the mobile application may be configured to provide a detailed indication of a progress of an injection sequence. For example, the mobile application may provide text, visual, and/or audible indications of progress with greater granularity than shown by LEDs, for example, as described herein previously.

In some embodiments, the mobile application may be configured to record and store a date and/or time of an injection. Based on the date and/or time of the injection, and a user's prescription information, the mobile application may be configured to automatically create a reminder for a subsequent injection. In some embodiments, upon completion of an injection, the mobile application may be configured to provide a notification to the user with positive feedback for adherence to a prescription regimen. In some embodiments, the mobile application may provide points and/or rewards for continued adherence.

In some embodiments, the mobile application may be configured to authenticate a user of the auto-injector 2 prior to use. For example, the mobile application, in connection with the user's mobile device, may use biometric identification, two-factor authentication, or any other suitable authentication protocol to confirm the identity of the user prior to an injection. Upon authentication of the user, the mobile application may cause the auto-injector to become activated or otherwise be unlocked. Such user authentication may inhibit misuse and/or waste of costly medicaments by persons other than an intended user.

In some embodiments, the mobile application may be configured to detect operating conditions of auto-injector 2. For example, the mobile application may be configured to detect a battery level of the device and in case of a low battery indication, the mobile application may be configured to provide a notification to the user indicative of a need to charge the device. In some embodiments, the mobile application may be configured to detect mechanical and/or electrical malfunctions of auto-injector 2 and convey such information to the user.

FIG. 18 shows an exemplary method 2000 according to the disclosure. Method 2000 may start at step 2002, where a user may position auto-injector 2 on her body so that tissue-engaging surface 4 contacts a skin surface. The user may position auto-injector 2 on her skin after removing locking component 1610, as described herein previously, thereby allowing touch sensor 1410 to detect proximity of the skin. Auto-injector 2 may be mounted in any suitable location, such as, e.g., the thigh, abdomen, shoulder, forearm, upper arm, leg, buttocks, or another suitable location. Auto-injector 2 may be secured to the skin by adhesive patch 12. The securement of auto-injector 2 at step 2002 may cause activating switch 1409, which extends outward from tissue-engaging surface 4, to be depressed and break a circuit. The breaking of the circuit may cause a signal to be sent to controller 1408 indicative that activating switch 1409 has been depressed. Alternatively, any other suitable mechanism may power on or otherwise activate auto-injector 2 before or after step 2002. Upon depression of activating switch 1409, auto-injector 2 may emit an audio tone and/or illuminate one or more LEDs (e.g., one or more LEDs of a first color, e.g., blue) to indicate depression of activating switch 1409.

Once auto-injector 2 is activated at step 2002, method 2000 may proceed to step 2004, where controller 1408 may determine whether tissue-engaging surface 4 is positioned on a skin surface. At step 2004, controller 1408 may receive a measurement from touch sensor 1410 indicating whether auto-injector 2 is positioned on skin or another surface. If controller 1408 determines that touch sensor 1410 is in contact with skin, for example, when a capacitance value received from touch sensor 1410 is within a predetermined range, method 2000 may proceed to step 2008. If controller 1408 determines that touch sensor is not in contact with skin, for example, if the capacitance measurement received from touch sensor 1410 indicates that auto-injector 2 is in contact with a non-skin surface like wood or metal, method 2000 may proceed to step 2006. At step 2006, auto-injector 2 may be placed into an error condition. In the error condition, an LED may be activated (e.g., a red LED) to indicate to the user that an error has occurred, or a message may be displayed on a display screen. In some examples, auto-injector 2 may need to be manually reset before an injection can be completed. In other examples, auto-injector 2 may loop back to step 2004, wherein controller 1408 continuously attempts to determine whether touch sensor 1410 is in contact with skin. Method 2000 also may require that touch sensor 1410 be in contact with skin during the entire injection. Thus, if at any point during the injection, controller 1408 determines that touch sensor 1410 is no longer in contact with skin, controller 1408 may stop the injection (e.g., by stopping further movement of translation mechanism 1366), may generate an error signal or message, and may retract needle 306 if it had been extended. By stopping the injection and retracting needle 306, a risk of dispensing the drug outside of the body (i.e. a wet injection) and/or needle stick injuries may be mitigated. Upon the determination of step 2004, auto-injector 2 may emit an audio tone and/or illuminate one or more LEDs to indicate that the auto-injector 2 is positioned on the skin surface. In one example, one or more additional LEDs of the first color may be illuminated at this stage to indicate further progress of the injection.

At step 2008, controller 1408 may send a signal to activate translation mechanism 1366. Once activated, translation mechanism 1366 may move toward second end 1306 of cartridge 1302 (referring to FIGS. 13 and 14), causing cartridge 1302 itself to move in the same direction. This may cause needle 308 to move in the opposing direction to access cartridge 1302 as set forth above. The movement of driver 1398 and needle 308 causes carrier 202 to move in the same direction, which sets forth the chain of events that ultimately deploys needle 306 into the user by the mechanisms set forth in FIGS. 5-11. Translation mechanism 1366 will continue to move toward second end 1306 until a desired amount of the drug contained within cartridge 1302 is dispensed into the user. Upon activation of the translation mechanism, auto-injector 2 may emit an audio tone and/or illuminate one or more LEDs to indicate that the injection is in progress. For example, yet additional LEDs of the first color may be illuminated as the injection progresses to give the user a visual indication of the progression.

Method 2000 may proceed to step 2010, where controller 1408 may determine whether the injection is complete. This determination may be based on interruption of beam 1430 by piston 1316 (as described with reference to FIGS. 4A, 13, and 14). That is, when beam 1430 is broken (not received by detector 1416), controller 1408 may determine that injection is complete. Once controller 1408 determines that the injection is complete, controller 1408 may send a signal to translation mechanism 1366 to reverse the direction of rotation of the lead screw, which may cause ramp 1500 to push against ramp 243 of stop 240, enabling retraction of needle 306 as discussed above with reference to FIG. 11. In one example, controller 1408 may institute a delay after receiving an indication that beam 1430 has been interrupted. The delay may be from, e.g., 0.1 to 60 seconds.

An additional end detection mechanism may be used instead of or in combination with the interruption-type sensor described above. For example, a current of the motor of translation mechanism 1366 may be utilized to determine whether an injection has been completed. That is, when piston 1316 reaches second end 1306 of cartridge 1302, the current on the motor will increase (e.g., as a result of piston 1316 engaging the end of cartridge 1302), signaling the expulsion of all or substantially all of the contents of cartridge 1302. One exemplary combination could include the use of beam 1430, where interruption of beam 1430 indicates that, e.g., 90 to 98 percent of the injection has been completed. Then, the current of the motor of translation mechanism 1366 could be analyzed to determine whether the remaining 2 to 10 percent of the injection has been completed. In another example, instead of using an optical switch, a delay from the initiation of the translation mechanism 1366 may be used by controller 1408 to determine when to reverse translation mechanism 1366. In one example, this delay may be from, e.g., about 1 to about 120 seconds, although other suitable times are also contemplated. In any event, the delay from initiation may be long enough to permit emptying of cartridge 1302. In still another example, beam 1430 may be used in combination with an encoder. The encoder may be configured to detect a position of piston 1316. If the encoder were used to detect the position of piston 1316 alone, a drive train issue could inhibit accurate detection. For example, piston 1316 may rotate when pushed by the lead screw. Such rotation may cause uncertainty as to actual position of piston 1316. When used in conjunction with beam 1430, however, controller 1408 may be configured to recalibrate the encoder in response to interruption of beam 1430. Such recalibration may allow controller 1408 to update the actual position of the encoder and resume accurate detection of the position of piston 1316 using the encoder.

Upon determination that the injection is complete, auto-injector 2 may emit an audio tone and/or illuminate one or more LEDs to indicate completion of the injection. In some examples, one or more LEDs of a second color (e.g., green) that is different from the first color may be illuminated to signal to the user that the injection is complete. In some examples, all of the LEDs of the device may be illuminated with the second color, and other indications also may be used. For example, all of the LEDs may be illuminated with the second color and may flash intermittently at the end of the injection.

In some examples, a timing of an injection procedure, measured from the initial activation of activating switch 1409 to retraction of needle 306 from the user after drug delivery, may be from about 20 seconds to about 90 seconds, or from about 25 seconds to about 60 seconds, from about 30 seconds to about 45 seconds, or less than or equal to about 120 seconds, or less than or equal to about 90 seconds, or less than or equal to about 60 seconds, or less than or equal to about 45 seconds, or less than or equal to about 30 seconds. Such timing represents a significant improvement over existing devices, for which the timing of an injection may be much longer and, in some cases, as long as about 9 minutes or even longer.

Method 2000 also may include additional steps. For example, method 2000 may include determining whether a drug within cartridge 1302 is too cold for delivery into the user, whether power source 1406 has enough energy to complete an injection, whether needle 306 has been prematurely deployed and/or retracted, whether the current of the motor of translation mechanism 1366 is in an appropriate range, and whether an injection procedure has extended beyond a maximum acceptable procedure time. When controller 1408 senses any of the above errors, it may communicate such errors to the user, and may end an ongoing injection by, e.g., halting or reversing translation mechanism 1366 and retracting needle 306 from the user. Auto-injector 2 may emit an audio tone and/or illuminate one or more LEDs indicative of any of the foregoing additional steps. For example, one or more LEDs of a third color (e.g., red) that is different than the first and second colors may be illuminated.

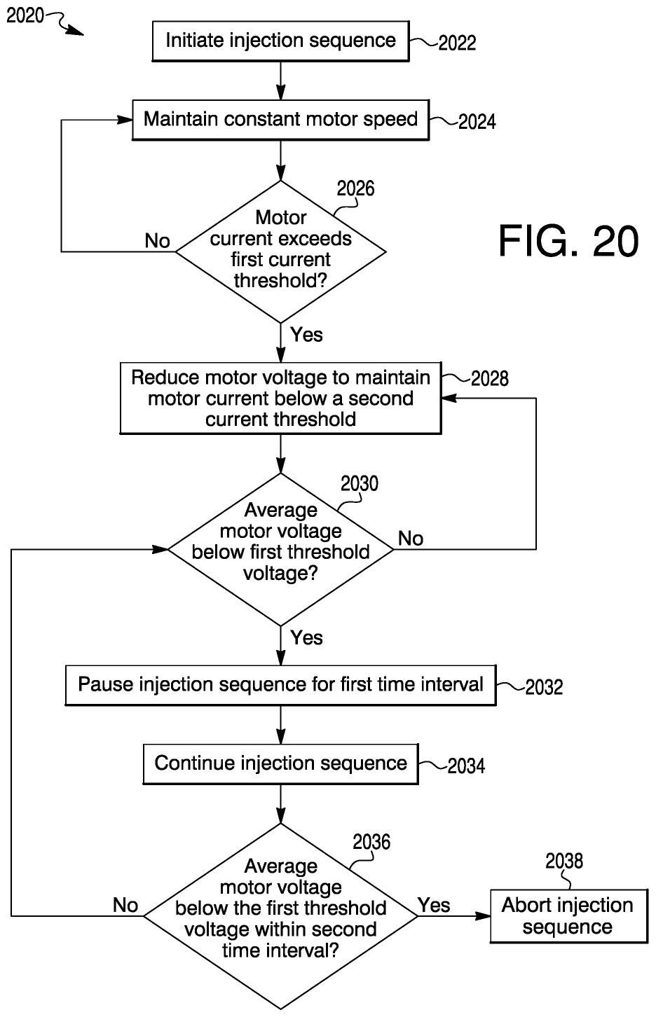

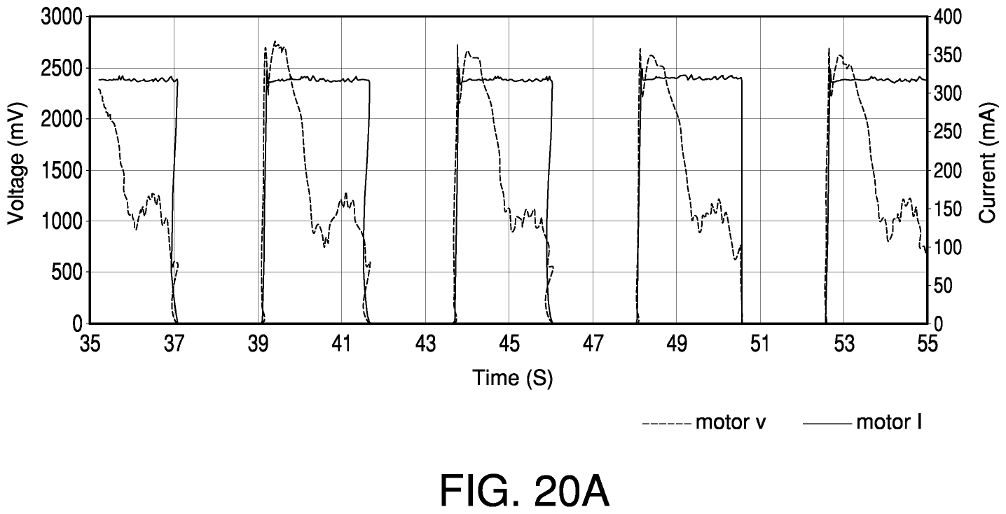

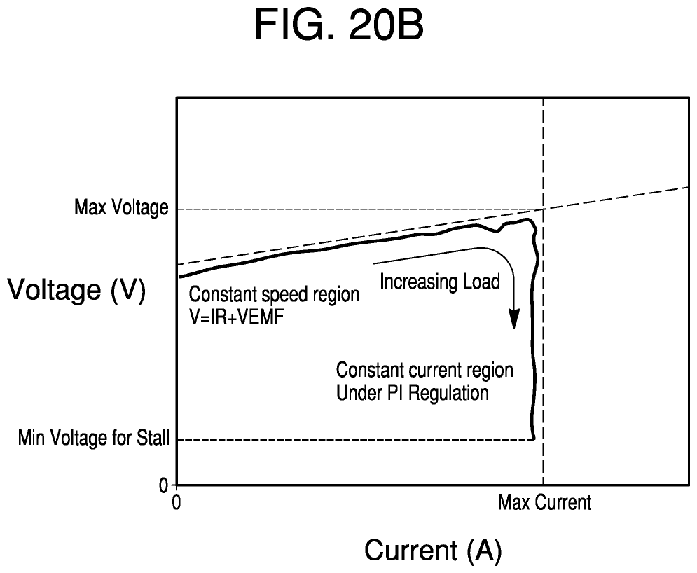

FIG. 20 shows an exemplary method 2020 of controlling a torque of the motor of translation mechanism 1366 and detecting when the motor stalls. At step 2022, controller 1408 may initiate an injection sequence. As described herein previously, an injection sequence may be initiated upon depressing activating switch 1409 against a user's skin and/or detecting the user's skin by touch sensor 1410. During the injection sequence, a voltage may be applied to the motor of translation mechanism 1366 to drive the motor.

At step 2024, as the injection sequence progresses, controller 1408 may maintain the motor of translation mechanism 1366 at a constant speed. The constant speed may be, for example, a rotational speed measured in revolutions per minute (RPM). Controller 1408 may maintain the motor at a constant speed by varying the voltage applied to the motor. For example, when a higher load is applied to the motor due to an obstruction, increased fluid pressure, increased component friction, or any other cause, controller 1408 may compensate for the increased load by increasing the voltage applied to the motor. Conversely, when a load applied to the motor is reduced, controller 1408 may compensate for the reduction in load by decreasing the voltage applied to the motor. Maintaining the motor at a constant speed may reduce a likelihood that the user experiences injection site pain. For example, maintaining the motor at a constant speed may prevent the bolus from become excessively large, thereby mitigating the risk of pain.

During the injection sequence, controller 1408 may monitor a current supplied to the motor. The motor current may be indicative of a torque generated by the motor. For example, a higher motor current may indicate a higher torque being generated by the motor. At step 2026, controller 1408 may determine whether the motor current exceeds a first current threshold. The first current threshold may be determined and/or set based on a maximum torque that may be safely generated by the motor. The maximum torque may be reached, for example, when the injection sequence is obstructed in some way. If controller 1408 determines that the motor current does not exceed the first current threshold, the method 2020 may revert to step 2024 and controller 1408 may continue to maintain the motor at a constant speed. If, on the other hand, controller 1408 determines that the motor current exceeds the first current threshold, method 2020 may proceed to step 2028.

At step 2028, controller 1408 may reduce the motor voltage to maintain the motor current below a second current threshold. In some embodiments, the second current threshold may be greater than the first current threshold and may more closely correlate to the maximum torque that may be safely generated by the motor. In some embodiments, the second current threshold may be less than, or the same as, the first current threshold. In the event that the injection sequence is obstructed, the motor speed may slow and the motor impedance may decrease. As the motor impedance decreases, a lower voltage may be required to maintain the motor current below the second current threshold. Controller 1408 may monitor an average motor voltage applied to the motor. The average motor voltage may be, for example, a time average.

Steps 2024 through 2028 of method 2020 may generally be illustrated by the graph depicted in FIG. 20B, in which a curve representing a relationship between the voltage applied to the motor of translation mechanism 1366 and the current consumed by the motor is plotted. The curve may be characterized by the following equation:

V=iR+V_{emf}

In the equation above, V is the voltage applied to the motor, i is a current consumed by the motor, R is a coil resistance of the motor, and V_{emf} is a back electromotive force that acts against the applied voltage at a given speed. As shown in FIG. 20B, the curve may include a constant speed region, in which the motor may be maintained a constant speed (step 2024). In the constant speed region, V_{emf} may remain approximately constant and the curve may be approximately linear.

As shown in FIG. 20B, as a load (i.e. a torque) acting on the motor of translation mechanism 1366 increases, the current consumed by the motor may increase. As the current approaches a Max Current, a voltage applied to the motor may be reduced, thereby maintaining the current below the Max Current (steps 2026 and 2028). The current consumed by the motor may be maintained below the Max Current using proportional integral (PI) regulation. As shown, there may be a minimum voltage for the motor below which the motor may stall.

Steps 2030 through 2038 of method 2020 may correspond to a control sequence for preventing stalling of the motor. FIG. 20A depicts a graph which may represent the voltage applied to the motor and the current consumed by the motor over time and in accordance with steps 2030 through 2038.

At step 2030, controller 1408 may determine whether the average motor voltage has decreased below a first threshold voltage. The average motor voltage decreasing below the first threshold voltage may indicate that the injection sequence is obstructed. If controller 1408 determines that the average motor voltage has not decreased below a first threshold voltage, method 2020 may revert to step 2028, at which controller 1408 may continue to maintain the motor current below the second current threshold. FIG. 20A illustrates five intervals during which controller 1408 may maintain the motor current at a constant value (e.g. below the second current threshold): between about 35 seconds and about 37 seconds, between about 39 seconds and about 41.5 seconds, between about 43.5 seconds and about 46 seconds, between about 48 seconds and about 50.5 seconds, and between about 52.5 seconds and about 55 seconds. As shown in FIG. 20A, the voltage applied to the motor during each interval may decrease, albeit with some fluctuations, to maintain the motor current below the second current threshold. Though the voltage during each interval in FIG. 20A is shown as decreasing, the voltage need not necessarily decrease to maintain the motor current below the second current threshold, but instead may stay flat in certain situations.

If, on the other hand, controller 1408 determines that the average motor voltage has decreased below the first threshold voltage, controller 1408 may cause the injection sequence to be paused for a first time interval. When causing the injection sequence to be paused, controller 1408 may cease applying voltage to the motor. In some embodiments, the first time interval may be 2 seconds, for example. FIG. 20A illustrates four such pauses: between about 37 seconds and about 39 seconds, between about 41.5 seconds and about 43.5 seconds, between about 46 seconds and about 48 seconds, and between about 50.5 seconds and about 52.5 seconds.

The first time interval may be sufficiently long to allow fluid pressure within auto-injector 2 to dissipate. The first time interval may also be sufficiently short such that the user may not be prompted to remove auto-injector 2 from the user's skin (e.g., the first time interval is set to be less than a typical reaction time of the user to falsely identify the end of the injection). The first time interval may further be indicated by illumination of one or more of the LEDs of the progress ring or another light within auto-injector 2 and visible by a user. The LEDs may be illuminated, for example, in a particular pattern or according to a particular color scheme to indicate the first time interval and that the injection sequence is paused rather than stopped.

After pausing the injection sequence, controller 1408 may continue the injection sequence at step 2034. To continue the injection sequence, controller 1408 may resume supplying voltage to the motor of translation mechanism 1366. At step 2036, controller 1408 may determine whether the average motor voltage has decreased below the first threshold voltage within a second time interval. The second time interval may be shorter than the first time interval and may be set and/or determined to be indicative of a confirmation that the injection sequence is obstructed. The second time interval may be, for example, about 0.9 seconds. If the motor voltage has not decreased below the first threshold voltage within the second time interval, method 2020 may revert to step 2030. If, on the other hand, controller 1408 determines that the motor voltage has decreased below the first threshold voltage within the second time interval, method 2020 may proceed to step 2038 at which controller 1408 may cause the injection sequence to be aborted.

In some embodiments, controller 1408 may perform step 2026 continuously as it performs steps 2028 to 2036. For example, controller 1408 may continue to determine whether the motor current exceeds the first current threshold as steps 2028 to 2036 are performed. If the motor current continues to exceed the first current threshold, method 2020 may proceed through steps 2028 to 2036 as described herein previously. In the event the motor current falls below the first current threshold, on the other hand, method 2020 may revert to step 2024 and controller 1408 may maintain the motor at a constant speed. In other words, if a high load on the motor, due to obstruction, high fluid pressure, or the like, dissipates during performance of steps 2028 to 2036, controller 1408 may simply revert to maintaining a constant motor speed rather than proceeding through any remaining steps unnecessarily.

Accordingly, method 2020 may allow controller 1408 to effectively distinguish between situations in which the needle may be partially blocked or a high friction force may be acting against the injection sequence, and situations in which the injection sequence is insurmountably obstructed. In the former situations, auto-injector 2 may have the ability to complete the injection sequence and the injection sequence may not be prematurely terminated. In the latter situations, auto-injector 2 may not have the ability to complete the injection sequence and the injection sequence may be appropriately terminated. In such situations, auto-injector 2 may emit an audio tone and/or illuminate one or more LEDs to indicate that the injection was terminated before completion. Method 2020 may further appropriately terminate an injection sequence in which the piston 1316 extends completely, indicating that the cartridge 1302 is empty. Method 2020 may further allow the auto-injector 2 to be used on an emergency basis if, for example, a user performs an injection without first warming up auto-injector 2 to decrease a viscosity of the medicament. Method 2020 may further allow an injection of a viscous medicament to proceed at a slower rate than the motor and gear reduction ratio may otherwise allow.

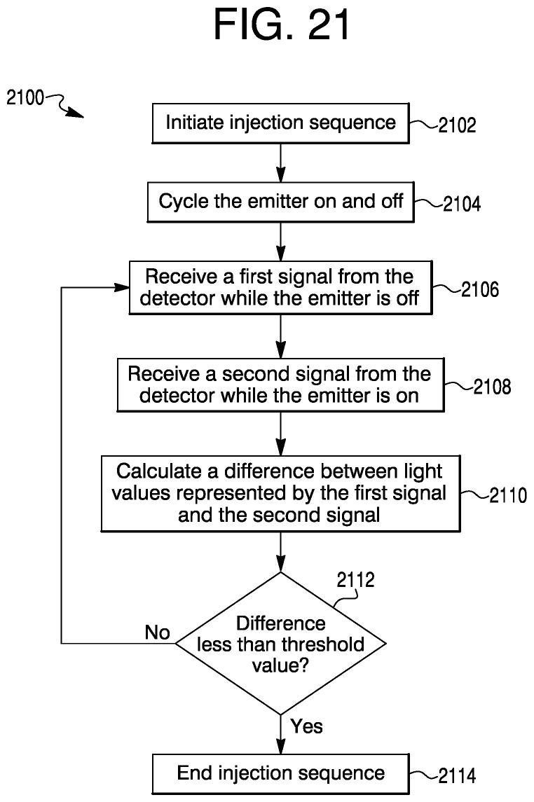

FIG. 21 shows an exemplary method 2100 of detecting an end of a dose of medicament using emitter 1414 and detector 1416. Method 2100 may be used, for example, to detect a time at which a full dose of medicament has been dispensed to a user and end the corresponding injection sequence.

At step 2102, controller 1408 may initiate an injection sequence. As described herein previously, an injection sequence may be initiated upon depressing activating switch 1409 against a user's skin and/or detecting the user's skin by touch sensor 1410. At step 2104, controller 1408 may cycle emitter 1414 on and off periodically. Emitter 1414 may be cycled on and off rapidly in a square wave pattern, such that emitter 1414 is turned off and on several times per second. Cycling emitter 1414 on and off may allow detector 1416 to be exposed to light produced by emitter 1414 in combination with ambient light, and also to ambient light alone.

At step 2106, controller 1408 may receive a first signal from detector 1416 corresponding to a time when emitter 1414 is off. The first signal may correspond to, and/or be indicative of, ambient light detected by the detector 1416. At step 2108, controller 1408 may receive a second signal from detector 1416 corresponding to a time when emitter 1414 is on. The second signal may correspond to, and/or be indicative of, light emitted by emitter 1414 in combination with ambient light as detected by the detector 1416.

At step 2110, controller 1408 may calculate a difference between a first light value represented by the first signal and a second light value represented by the second signal. The difference may be indicative of how much light detected by detector 1416 is attributable to light emitted by emitter 1414 as opposed to ambient light. At step 2112, controller 1408 may determine whether the difference is less than a threshold value. If controller 1408 determines that the difference is not less than a threshold value, method 2100 may revert to step 2106. If, on the other hand, controller 1408 determines that the difference is less than the threshold value, controller 1408 may end the injection sequence at step 2114.

Accordingly, method 2100 may be used to reduce the impact of ambient light when detecting an end of a dose of medicament. Specifically, method 2100 may address a situation in which light from emitter 1414 is blocked from reaching the detector 1416 indicating an end of a dose, yet ambient light is able to reach detector 1416 and create a false negative reading indicating that an end of dose has not been reached.

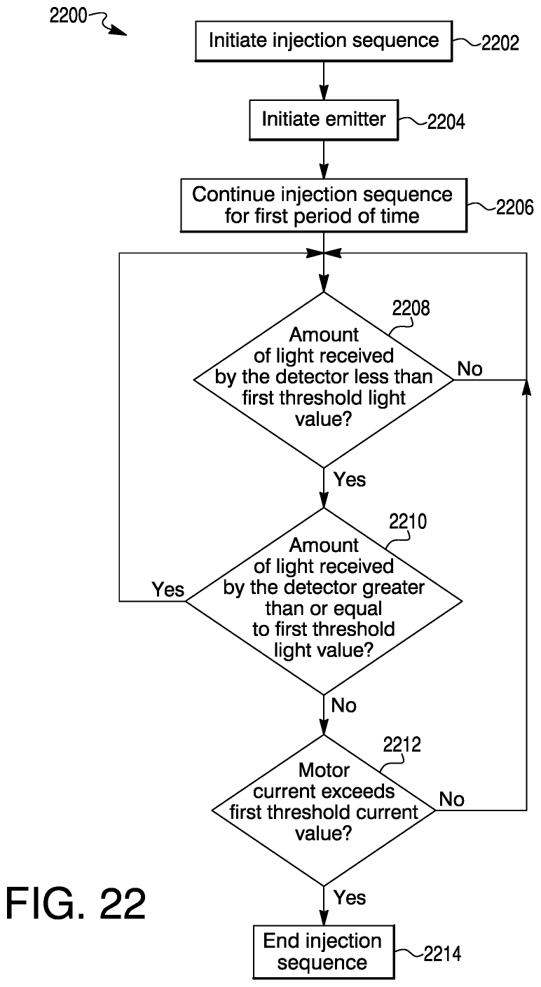

FIG. 22 shows another exemplary method 2200 of detecting an end of a dose of medicament using emitter 1414 and detector 1416. Method 2200 may be used, for example, to detect a time at which a full dose of medicament has been dispensed to a user and end the corresponding injection sequence.

At step 2202, controller 1408 may initiate an injection sequence. As described herein previously, an injection sequence may be initiated upon depressing activating switch 1409 against a user's skin and/or detecting the user's skin by touch sensor 1410. At step 2204, controller 1408 may initiate emitter 1414 or otherwise cause emitter 1414 to emit light.

At step 2206, controller 1408 may cause the injection sequence to continue for a first period of time. The first period of time may be a predetermined period of time corresponding to a duration in which a full dose cannot possibly be, or is unlikely to be, dispensed. For example, the first period of time may be between about 20% and 50% of the total injection time. During the first period of time, controller 1408 is not able to interrupt the injection sequence in response to a signal received from detector 1416 (but could still interrupt the injection sequence due to obstructions or stalling as discussed with reference to FIG. 20).

At step 2208, after the end of the first period of time, controller 1408 may determine whether an amount of light received by detector 1416 is less than a first threshold light value. Controller 1408 may make the determination based on a signal received from detector 1416 indicative of light received by detector 1416. The first threshold light value may correspond to an amount of light received by detector 1416 at the end of a dose. If controller 1408 determines that the amount of light received by detector 1416 is not less than the first threshold light value, controller 1408 may continue the injection sequence and method 2200 may otherwise remain at step 2208. If, on the other hand, controller 1408 determines that the amount of light received by detector 1416 is less than the first threshold light value, the method may proceed to step 2210.

At step 2210, controller 1408 may determine whether the amount of light received by detector 1416 is greater than or equal to the first threshold light value. If controller 1408 determines that the amount of light received by detector 1416 has risen to or above the first threshold light value, controller 1408 may continue the injection sequence and method 2200 may revert to step 2208. If, on the other hand, controller 1408 determines that the amount of light received by detector 1416 has remained less than the first threshold light value, the method may proceed to step 2212. Step 2210 may in effect enable controller 1408 to “clear” the injection sequence of anomalous interruptions of the light received by the detector, which may be caused by an air bubble within cartridge 1302 that blocks the path of light between emitter 1414 and detector 1416, for example, provided the amount of light subsequently meets or exceeds the first threshold light value.

At step 2212, controller 1408 may determine whether the motor current exceeds a first threshold current value. The first threshold current value may be determined and/or set based on a current indicative of an end of the injection sequence. The first current threshold value may be set, for example, based on a current indicative of piston 1316 reaching second end 1306 of cartridge 1302. If controller 1408 determines that the motor current does not exceed the first threshold current value, controller 1408 may continue the injection sequence and method 2200 may revert to step 2208. If, on the other hand, controller 1408 determines that the motor current exceeds the first threshold current value, method 2200 may proceed to step 2214 at which controller 1408 may cause the injection sequence to end.

Method 2200 may accordingly allow for accurate identification of the end of an injection sequence by identifying an instant in which both the light received by detector 1416 and the motor current are indicative of an end of the dose. By performing steps 2208, 2210, and 2212 sequentially, false identifications of the end of the dose due to either anomalous interruptions of light or anomalous high current events alone may be mitigated. Method 2200 may specifically reduce the impact of bubbles within cartridge 1302 on detection of the end of a dose of medicament.

FIG. 23 shows an exemplary method 2300 of operating activating switch 1409 of auto-injector 2 according to the disclosure. In particular, FIG. 23 depicts an exemplary sequence of positions of activating switch 1409 and corresponding functions of auto-injector 2.

Initially, at step 2302, auto-injector 2 may be disposed within a packaging such that plunger 1450 is in a depressed state and auto-injector 2 is in a low-power sleep mode. In some embodiments, during manufacturing auto-injector 2 may be programmed in an awake or active state. In some embodiments, if plunger 1450 is depressed for a predetermined period of time following programming, such as when auto-injector is placed in the packaging, auto-injector 2 may be configured to transition to the low-power sleep mode. The predetermined period of time may be any suitable period of time, such as 60 minutes, 30 minutes, 15 minutes, 10 minutes, 5 minutes, 2 minutes, or any other suitable period. Auto-injector 2 may be sealed in the packaging such that the packaging indicates that the auto-injector 2 has not been previously used. The packaging may be made from any suitable material, including paper, cardboard, plastic, cellophane, and the like. The packaging may press against plunger 1450 such that plunger 1450 is flush or nearly flush with housing 3 of auto-injector 2 and plunger 1450 is blocked from extending outwardly from auto-injector 2. With plunger 1450 in the depressed state, the circuit associated with activating switch 1409 may be open, thereby maintaining the auto-injector 2 in the low-power sleep mode.

At step 2304, auto-injector 2 may be removed from the packaging such that plunger 1450 is no longer depressed by the packaging and plunger 1450 may extend outwardly from the auto-injector 2. As plunger 1450 transitions from the depressed state to the free or extended state, plunger flange 1454 may contact or otherwise depress plunger switch 1448, thereby completing the circuit associated with activating switch 1409.

At step 2306, in response to the circuit associated with activating switch 1409 being completed, auto-injector may transition from the low-power sleep mode to an active mode. In the active mode, auto-injector 2 may calibrate touch sensor 1410. Auto-injector 2 may calibrate touch sensor 1410 by detecting a value or measurement of the touch sensor 1410 in ambient air, i.e. not against a user's skin. Auto-injector 2 may perform such calibration during a predetermined time period after auto-injector 2 is removed from the packaging (in some cases immediately after removal) so that such calibration occurs before a user may expose their skin to touch sensor 1410. In the active mode, auto-injector 2 may further detect whether emitter 1414 and/or detector 1416 are functioning properly, detect whether a needle is positioned properly, detect whether the motor of translation mechanism 1366 is responsive and/or operational, and/or perform any other suitable status tests. Auto-injector 2 may detect the positioning of the needle, for example, using a switch or detector configured to report the position of the needle to the controller 1408. In the active mode, auto-injector 2 may further illuminate one or more backlights to allow a user to inspect a vial and/or a drug contained in the vial through transparent window 50. In the active mode, auto-injector 2 may further display any other indication that auto-injector 2 is ready to be used.

At step 2308, auto-injector 2 may be placed against a user's skin such that plunger 1450 is depressed into auto-injector 2. Upon plunger 1450 being depressed, the circuit associated with activating switch 1409 may transition to an open state. As described above with reference to FIG. 18 and method 2000, auto-injector 2 may further detect contact with skin using touch sensor 1410. In response to depression of plunger 1450 and detection of contact with skin, auto-injector 2 may initiate an injection sequence at step 2310. The injection sequence may be a sequence resulting in injection of the user with a medicament, as described herein previously.

At step 2312, auto-injector 2 may be removed from the user's skin and plunger 1450 may again extend outwardly from auto-injector 2. Upon plunger 1450 extending outwardly, the circuit associated with activating switch 1409 may transition from the open state to the closed state. In response, auto-injector 2 may end the injection sequence at step 2310 and, for example, initiate retraction of the patient needle by reversing the motor. Auto-injector 2 may initiate retraction of the needle if the injection sequence has proceeded to completion or if the auto-injector has prematurely or accidentally been removed from the skin to prevent wet injection. Alternatively, in some embodiments, controller 1408 may determine whether a value received from touch sensor 1410 is indicative of the auto-injector 2 remaining in contact with the user's skin. If the value received by controller 1408 is indicative of the auto-injector 2 remaining in contact with the user's skin, auto-injector 2 may pause the injection sequence, thereby preventing wet injection. If the plunger 1450 is again depressed, thereby placing the circuit associated with activating switch 1409 in the open state, auto-injector 2 may resume the injection sequence.

According to the foregoing method 2300, activating switch 1409 may serve to keep auto-injector 2 in a lower-power sleep mode when in the packaging, transition auto-injector 2 to an active mode upon removal from the packaging, indicate when auto-injector 2 has been placed against a user's skin for an injection sequence, and indicate when auto-injector 2 has been removed from the user's skin at the end of an injection sequence. Moreover, a signal from activating switch 1409 may be cross-checked against a signal from touch sensor 1410 to more accurately determine whether auto-injector 2 has been removed from the user's skin, or whether, for example, an inadvertent or minor movement of auto-injector occurred.

It should be understood that steps of one or more of the various methods described herein may be combined in certain embodiments. Furthermore, in certain embodiments, fewer than all of the steps of a method described herein may be performed and/or additional steps not described herein may be performed. Moreover, the steps described herein need not necessarily be performed in the exact order presented.

Notably, reference herein to “one embodiment,” or “an embodiment” means that a particular feature, structure, or characteristic described in connection with the embodiment may be included, employed and/or incorporated in one, some or all of the embodiments of the present disclosure. The usages or appearances of the phrase “in one embodiment” or “in another embodiment” in the specification are not referring to the same embodiment, nor are separate or alternative embodiments necessarily mutually exclusive of one or more other embodiments, nor limited to a single exclusive embodiment. The same applies to the terms “implementation,” and “example.” The present disclosure are neither limited to any single aspect nor embodiment thereof, nor to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein.

Further, as indicated above, an embodiment or implementation described herein as “exemplary” is not to be construed as preferred or advantageous, for example, over other embodiments or implementations; rather, it is intended convey or indicate the embodiment or embodiments are example embodiment(s).

The present disclosure is further described by the following non-limiting items:

Item 1. An injection device, comprising:

a housing;

a container disposed within the housing, the container enclosing a fluid and having a first end and a second end;

a conduit movable relative to the container, wherein the conduit is not in fluid communication with the fluid enclosed by the container while in a first position, and is in fluid communication with the fluid enclosed by the container and configured to deliver the fluid from the container to a patient while in a second position;

a lock that is removable from the housing, the lock having a first portion and a second portion, wherein:
in a first configuration where the lock is coupled to the housing, the first portion of the lock is disposed exterior of the housing and the second portion of the lock is disposed within the housing between the container and the conduit;
in the first configuration, the conduit is prevented from moving into fluid communication with the fluid enclosed by the container by the second portion of the lock; and
in a second configuration where the lock is removed from the injection device, the conduit is able to move into fluid communication with the fluid enclosed by the container.

Item 2. The injection device of item 1, further including:

an adhesive disposed exterior of the housing; and

a cover disposed over the adhesive, wherein the cover is removable from the injection device to expose the adhesive,

wherein the lock is coupled to the cover such that the lock is removed from the injection device when the cover is removed from the injection device.

Item 3. The injection device of item 1, further including a controller and a skin sensor coupled to the controller, wherein:

the skin sensor is configured to detect when skin is in contact with an outer surface of the housing adjacent to the skin sensor;

the controller is configured to cause the conduit to move into fluid communication with the fluid enclosed by the container; and

when the lock is coupled to the housing in the first configuration, the first portion of the lock is configured to prevent skin from contacting the outer surface of the housing adjacent to the skin sensor.

Item 4. The injection device of item 1, wherein, while in the first configuration, the lock is configured to prevent inadvertent activation of the injection device when the injection device is dropped or subject to vibration.

Item 5. An injection device, comprising:

a housing;

a plunger coupled to the housing and movable relative to the housing;

one or more electronics components used during an injection performed by the injection device, the one or more electronics components being formed within an electrical circuit, wherein:
in a first configuration, a first portion of the plunger is disposed within the housing, the electrical circuit is open, and the one or more electronics components are in a low-power sleep mode;
in a second configuration, the plunger moves outward relative to the housing, and the first portion of the plunger extends exterior of the housing; and
in the second configuration, the electrical circuit is closed, and the one or more electronics components are transitioned from the low-power sleep mode, to an active mode.

Item 6. The injection device of item 5, further comprising a biasing member movable from a resting position to a stressed position, wherein:

in the first configuration, the biasing member is in the stressed position; and

in the second configuration, the biasing member is in the resting position.

Item 7. The injection device of item 6, further including a stop configured to maintain the biasing member in the stressed position and the plunger in the first configuration when the stop is in a blocking position, wherein movement of the stop from the blocking position allows the biasing member to move to the resting position and the plunger to move to the second configuration.

Item 8. The injection device of item 7, wherein the stop includes a packaging of the injection device.

Item 9. The injection device of item 5, wherein the one or more electronics components include a switch, and transition of the plunger from the first configuration to the second configuration moves the switch to close the electrical circuit.

Item 10. The injection device of claim 5, wherein:

the plunger is movable from the second configuration toward the housing to a third configuration; and

the injection device is configured to initiate an injection by the injection device only after the plunger is moved to the third configuration and the one or more electronics components are in the active mode.

Item 11. The injection device of item 10, wherein:

the one or more electronics components include a skin sensor and the injection device further includes a controller configured to initiate the injection by the injection device;

the skin sensor is configured to detect a presence of skin in contact with an outer surface of the housing adjacent to the skin sensor only after the plunger has moved to the third configuration; and

the controller is configured to initiate the injection only after detection of skin by the skin sensor.

Item 12. The injection device of item 11, further including a motor configured to dispense medicament from the injection device during the injection, wherein:

during the injection, the plunger is movable from the third configuration, away from the housing, to a fourth configuration; and

the controller is configured to cease operation of the motor while the motor is operating and the controller senses the plunger has moved from the third configuration to the fourth configuration.

Item 13. The injection device of item 12, wherein the controller is configured to:

determine that skin has remained in contact with the outer surface of the housing adjacent to the skin sensor, while the plunger is in the fourth configuration; and

resume operation of the motor when the plunger moves from the fourth configuration to the third configuration, after the controller determines that skin has remained in contact with the outer surface of the housing adjacent to the skin sensor while the plunger was in the fourth configuration.

Item 14. An injection device, comprising:

a housing, wherein the housing includes a curved bottom surface that is concave when viewed from a point external to the housing that is closer to the bottom surface of the housing than a top surface of the housing;

a circuit board positioned adjacent to the bottom surface of the housing, wherein the circuit board includes a skin sensor configured to sense a presence of skin in contact with the bottom surface of the housing; and

a controller coupled to the circuit board, wherein the controller is configured to initiate an injection by the injection device only after the skin sensor senses the presence of skin in contact with the bottom surface of the housing.

Item 15. The injection device of item 14, wherein the skin sensor is configured to sense the presence of skin in contact with the bottom surface of the housing without skin directly contacting the skin sensor.

Item 16. The injection device of item 14, wherein the skin sensor is a capacitance sensor.

Item 17. The injection device of item 14, wherein:

the housing further includes an opening in the curved bottom surface;

the injection device further includes a needle extendable out of the housing through the opening; and

the skin sensor is positioned adjacent to the opening.

Item 18. The injection device of item 14, wherein the circuit board is the only circuit board disposed within the housing.

Item 19. A method of manufacturing an injection device, the method comprising:

depositing a first material onto a mold, the first material having a first opacity;

depositing a second material around the mold and the first material, the second material having a second opacity that is higher than the first opacity; and

positioning a container enclosing a medicament within the injection device and adjacent to a first portion of the injection device formed by the first material.

Item 20. The method of item 19, further including positioning one or more LEDs within the injection device and adjacent to one or more second portions of the injection device, separate from the first portion of the injection device, the one or more second portions of the injection device being formed by the first material.

Item 21. An injection device, comprising:

a container disposed within the housing, the container having a first end and a second end;

a piston configured to move from the first end of the container toward the second end of the container to dispense a medicament from the container;

a drive member configured to drive the piston through the container;

an emitter configured to emit a beam of light toward the container;

a detector positioned on an opposing side of the container from the emitter, wherein the detector is configured to receive the beam of light emitted from the emitter; and

a controller coupled to the drive member, the emitter, and the detector, the controller being configured to:
receive a first signal from the detector while the emitter is off, the first signal corresponding to an ambient level of light surrounding the injection device;
receive a second signal from the detector while the emitter is on;
calculate a difference between light values represented by the first signal and the second signal; and
cease operation of the drive member when the difference is less than a threshold value.

Item 22. An injection device, comprising:

a container disposed within the housing, the container having a first end and a second end;

a piston configured to move from the first end of the container toward the second end of the container to dispense a medicament from the container;

a drive member configured to drive the piston through the container;

an emitter configured to emit a beam of light toward the container;

a detector positioned on an opposing side of the container from the emitter, wherein the detector is configured to receive the beam of light emitted from the emitter; and

a controller coupled to the drive member, the emitter, and the detector, the controller being configured to:
initiate the drive member and the emitter;
receive a first signal from the detector while the emitter is on, the first signal being representative of an amount of light received by the detector;
allow for continued operation of the drive member for a first period of time immediately after initiation of the drive member; and
cease operation of the drive member upon determining (1) that the amount of light received by the detector is less than a first threshold light value and (2) before the amount of light received by the detector subsequently rises to or above the first threshold light value, that a current of the drive member is greater than a first threshold current value.

Item 23. The injection device of item 22, wherein the controller is further configured to:

continue operation of the drive member if the determined amount of light received by the detector is less than the first threshold value and, before the current of the drive member exceeds the first threshold current value, the amount of light received by the detector rises to or above the first threshold light value.

Item 24. An injection device, comprising:

a container disposed within the housing, the having a first end and a second end;

a piston configured to move from the first end of the container toward the second end of the container to dispense a medicament from the container;

a drive member configured to drive the piston through the container; and

a controller coupled to the drive member, the controller being configured to:
maintain a speed of the drive member until a current of the drive member exceeds a first threshold; and
after the current of the drive member exceeds the first threshold, reduce a voltage of the drive member to maintain the current of the drive member below a second threshold that is greater than or equal to the first threshold.

Item 25. The injection device of item 1, further including a controller and a skin sensor coupled to the controller, wherein:

the skin sensor is configured to detect when skin is in contact with an outer surface of the housing adjacent to the skin sensor;

the controller is configured to cause the conduit to move into fluid communication with the fluid enclosed by the container; and

the injection device further comprises a removable cover configured to prevent skin from contacting the outer surface of the housing adjacent to the skin sensor.

Item 26. The method of item 19, further comprising:

positioning a conduit movable relative to the container within the injection device such that the conduit is not in fluid communication with the fluid enclosed by the container;

wherein the first portion includes a first transparent window through which an interior of the injection device is visible; and

wherein the container is visible through the transparent window.

Item 27. The method of item 26, wherein the first portion further includes a plurality of second transparent windows arranged on a top surface of the injection device, wherein at least one LED is visible through each of the plurality of second transparent windows; and

wherein the first transparent window and the plurality of second transparent windows are collectively formed of a contiguous portion of the first material.

What is claimed is:

1. An injection device, comprising:
a housing;
a container disposed within the housing, the container enclosing a fluid and having a first end and a second end;
a conduit movable relative to the container, wherein the conduit is not in fluid communication with the fluid enclosed by the container while in a first position, and is in fluid communication with the fluid enclosed by the container and configured to deliver the fluid from the container to a patient while in a second position;
a lock that is removable from the housing, the lock having a first portion and a second portion, wherein:
in a first configuration where the lock is coupled to the housing, the first portion of the lock is disposed exterior of the housing and the second portion of the lock is disposed within the housing between the container and the conduit;
in the first configuration, the conduit is prevented from moving into fluid communication with the fluid enclosed by the container by the second portion of the lock; and
in a second configuration where the lock is removed from the injection device, the conduit is able to move into fluid communication with the fluid enclosed by the container.

2. The injection device of claim 1, further including:
an adhesive disposed exterior of the housing; and
a cover disposed over the adhesive, wherein the cover is removable from the injection device to expose the adhesive,
wherein the lock is coupled to the cover such that the lock is removed from the injection device when the cover is removed from the injection device.

3. The injection device of claim 1, further including a controller and a skin sensor coupled to the controller, wherein:
the skin sensor is configured to detect when skin is in contact with an outer surface of the housing adjacent to the skin sensor;
the controller is configured to cause the conduit to move into fluid communication with the fluid enclosed by the container; and
when the lock is coupled to the housing in the first configuration, the first portion of the lock is configured to prevent skin from contacting the outer surface of the housing adjacent to the skin sensor.

4. The injection device of claim 1, wherein, while in the first configuration, the lock is configured to prevent inadvertent activation of the injection device when the injection device is dropped or subject to vibration.

5. An injection device, comprising:
a housing;
a plunger coupled to the housing and movable relative to the housing;
one or more electronics components used during an injection performed by the injection device, the one or more electronics components being formed within an electrical circuit, wherein:
in a first configuration, a first portion of the plunger is disposed within the housing, the electrical circuit is open, and the one or more electronics components are in a low-power sleep mode;
in a second configuration, the plunger moves outward relative to the housing, and the first portion of the plunger extends exterior of the housing; and
in the second configuration, the electrical circuit is closed, and the one or more electronics components are transitioned from the low-power sleep mode, to an active mode.

6. The injection device of claim 5, further comprising a biasing member movable from a resting position to a stressed position, wherein:
in the first configuration, the biasing member is in the stressed position; and
in the second configuration, the biasing member is in the resting position.

7. The injection device of claim 6, further including a stop configured to maintain the biasing member in the stressed position and the plunger in the first configuration when the stop is in a blocking position, wherein movement of the stop from the blocking position allows the biasing member to move to the resting position and the plunger to move to the second configuration.

8. The injection device of claim 7, wherein the stop includes a packaging of the injection device.

9. The injection device of claim 5, wherein the one or more electronics components include a switch, and transition of the plunger from the first configuration to the second configuration moves the switch to close the electrical circuit.

10. The injection device of claim 5, wherein:
the plunger is movable from the second configuration toward the housing to a third configuration; and
the injection device is configured to initiate an injection by the injection device only after the plunger is moved to the third configuration and the one or more electronics components are in the active mode.

11. The injection device of claim 10, wherein:
the one or more electronics components include a skin sensor and the injection device further includes a controller configured to initiate the injection by the injection device;
the skin sensor is configured to detect a presence of skin in contact with an outer surface of the housing adjacent to the skin sensor only after the plunger has moved to the third configuration; and
the controller is configured to initiate the injection only after detection of skin by the skin sensor.

12. The injection device of claim 11, further including a motor configured to dispense medicament from the injection device during the injection, wherein:
during the injection, the plunger is movable from the third configuration, away from the housing, to a fourth configuration; and
the controller is configured to cease operation of the motor while the motor is operating and the controller senses the plunger has moved from the third configuration to the fourth configuration.

13. The injection device of claim 12, wherein the controller is configured to:
determine that skin has remained in contact with the outer surface of the housing adjacent to the skin sensor, while the plunger is in the fourth configuration; and
resume operation of the motor when the plunger moves from the fourth configuration to the third configuration, after the controller determines that skin has remained in contact with the outer surface of the housing adjacent to the skin sensor while the plunger was in the fourth configuration.

14. An injection device, comprising:
a housing, wherein the housing includes a curved bottom surface that is concave when viewed from a point external to the housing that is closer to the bottom surface of the housing than a top surface of the housing;
a circuit board positioned adjacent to the bottom surface of the housing, wherein the circuit board includes a skin sensor configured to sense a presence of skin in contact with the bottom surface of the housing; and
a controller coupled to the circuit board, wherein the controller is configured to initiate an injection by the injection device only after the skin sensor senses the presence of skin in contact with the bottom surface of the housing.

15. The injection device of claim 14, wherein the skin sensor is configured to sense the presence of skin in contact with the bottom surface of the housing without skin directly contacting the skin sensor.

16. The injection device of claim 14, wherein the skin sensor is a capacitance sensor.

17. The injection device of claim 14, wherein:
the housing further includes an opening in the curved bottom surface;
the injection device further includes a needle extendable out of the housing through the opening; and
the skin sensor is positioned adjacent to the opening.

18. The injection device of claim 14, wherein the circuit board is the only circuit board disposed within the housing.
